(19) United States
(12) Patent Application Publication
(10) Pub. No.: US 2025/0032372 A1
(43) Pub. Date: Jan. 30, 2025

(54) MACROSCOPIC DISPERSION WITH A DISPERSED FATTY PHASE HAVING HIGH CATIONIC POLYMER AND PIGMENT CONTENT

(71) Applicant: CAPSUM, MARSEILLE (FR)

(72) Inventor: Julie BACON, MARSEILLE (FR)

(21) Appl. No.: 18/712,621

(22) PCT Filed: Nov. 22, 2022
(86) PCT No.: PCT/EP2022/082873

§ 371 Date: May 22, 2024

(30) Foreign Application Priority Data
Nov. 24, 2021 (FR) FR2112460

(51) Int. Cl. A61K 8/11; A61Q 1/02

(52) U.S. Cl. CPC A61K 8/11; A61Q 1/02; A61K 2800/33; A61K 2800/41; A61K 2800/43; A61K 2800/5424; A61K 2800/5426; A61K 2800/594; A61K 2800/63; A61K 2800/652; A61K 2800/805

(57) ABSTRACT

The disclosure relates in general to macroscopic dispersions having high cationic polymer and pigment content, as well as to uses thereof in the cosmetic domain, and in particular to the uses thereof as a makeup composition, especially as foundation.

The present disclosure relates in general to dispersions with high contents of cationic polymer and pigments, and to the uses thereof in the field of cosmetics, and in particular the uses thereof as make-up composition, particularly a foundation.

One of the chief objectives in cosmetics is to improve the outer appearance of the skin, in particular of the face. In general, foundations are used to improve the characteristics or to hide the imperfections of the skin. Having regard to their powdery and insoluble nature, pigments are difficult to integrate in the dispersed phase of dispersions.

It therefore often proves complicated to add high contents of pigments to emulsified systems, in particular to the dispersed phases, without deteriorating the stability and sensorial experience thereof and the quality of the film deposited on the keratin material and in particular on the skin. Additionally, it is difficult to reconcile opposing requirements of technical performance in one same composition, such as coverage and the sensation of freshness even hydration. These problems are further heightened if a dispersion is considered comprising a dispersed phase in the form of droplets of macroscopic size.

Therefore, for foundation make-up, the emulsifying systems given priority are chiefly invert emulsions having pigments in the continuous phase on account of the good level of coverage and homogeneous appearance they provide compared with direct emulsions. Their weakness on the other hand is a strong oily and tacky sensation and/or lack of freshness and naturalness, and hence lack of lightweight property for the textures obtained. The few emulsifying systems on the market of «direct emulsion» type comprise pigments generally included in a continuous aqueous phase imparting thereto poor resistance to perspiration and humidity.

The Applicant has succeeded however in overcoming these drawbacks by proposing compositions in patent application WO2019053236 in the form of macroscopic dispersions able to impart long-lasting visual results to the skin with a sensation of lightness, freshness, and good hydration on application, this expected visual result preferably being coverage of colour imperfections and/or surface imperfections that is flawless.

Nevertheless, the Applicant has observed the presence of instabilities regarding these macroscopic dispersions and the microfluidic formation method thereof, in particular in terms of sphericity and/or mechanical strength of the droplets of the dispersed phase, the more so when the dispersion has high contents of dispersed fatty phase and/or when the dispersed fatty phase comprises high contents of pigments. These drawbacks hinder access to pigmented macroscopic dispersions having high percentages of pigmented fatty phase, and in particular higher than 10% relative to the total weight of the dispersion. Without wishing to be bound by any theory, the Applicant is of the opinion that these drawbacks can result from interaction between the pigments and the cationic polymer, amodimethicone in particular, this cationic polymer then being less available to ensure formation of the shell.

There remains a need therefore for macroscopic dispersions having a pigmented dispersed fatty phase which first is based on a reliable and robust production method and secondly comprises droplets having improved sphericity and mechanical strength whilst maintaining the advantageous properties thereof in terms of coverage of colour imperfection, sensory feel of freshness, hydration, and lightweight application, without any sensation of oiliness, tackiness, lack of slip or smoothness.

The improvement in the mechanical strength of the droplets of macroscopic dispersions without deteriorating comfort and sensorial experience on application is constantly sought after.

Unexpectedly the inventors have found that it is possible to meet the aforementioned objectives with a dispersion of the invention.

More specifically, the inventors have found that it is possible to accede to pigmented macroscopic dispersions having improved properties in terms of sphericity and mechanical strength of the droplets, provided that use is made of a dispersion in which the droplets have a shell comprising at least one anionic polymer and at least one cationic polymer, and in which the content of cationic polymer(s) is adjusted and more particularly is significantly increased.

The invention therefore concerns a dispersion comprising a fatty phase in the form of droplets dispersed in a continuous aqueous phase preferably in gel form, the dispersed phase and the continuous phase being non-miscible with each other at ambient temperature and atmospheric pressure, wherein the droplets comprise at least one shell and pigments, said shell being formed of at least one anionic polymer comprising at least one carboxylic acid function, and of at least one cationic polymer comprising at least two amine functions, characterized in that the quantity of amine functions contributed by the cationic polymer, in the fatty phase, is between 10.8 μmol and 32.4 μmol per gram of fatty phase.

As follows from the examples below, the inventors have observed that a dispersion of the invention is advantageous in terms of sphericity and mechanical strength of the droplets of the dispersed fatty phase.

This observation is all the more surprising and unexpected since WO2017046299 on the contrary teaches that the mechanical strength of the droplets of a macroscopic dispersion is improved in the presence of low contents of cationic polymer(s) relative to the weight of the dispersed fatty phase. This teaching arises from the identified optimal quantities of amine functions contributed by the cationic polymer in the dispersed fatty phase, namely less than 10.5 μmol per gram of dispersed fatty phase and preferably between 0.8 μmol and 2 μmol per gram of dispersed fatty phase.

Additionally, the inventors have observed that these advantages are not detrimental to the qualities of the dispersion of the invention which maintains the properties thereof in terms of long wear, flawless coverage of colour imperfections and/or of surface imperfections, combined with a sensation of lightness, freshness, and hydration on application and hence without any sensation of oiliness and/or tackiness and having smooth slip. A dispersion of the invention therefore continues to provide a unique colour shade on application, with a progressive or unfolding make-up result.

By «unique/improved colour shade», it is meant to designate a dispersion of the invention which, on application onto keratin material and in particular the skin, the type and content of pigment(s) being equivalent, forms a film on said keratin material having a darker shade than a conventional foundation composition, in particular in emulsion form.

By «progressive or unfolding make-up result», it is meant to designate a dispersion of the invention which, on application onto keratin material and in particular the skin, forms a film having colouring that is not or scarcely intense, this intensity gradually increasing over a short period of time i.e. a period of time longer than 15 seconds, preferably longer than 30 seconds, and shorter than 120 seconds, even shorter than 90 seconds and in particular shorter than 60 seconds.

In short, the unique make-up properties of a dispersion of the invention compared with those observed with a dispersion according to WO2019053236 remain.

The dispersion of the invention has the advantage of being stable, in particular over time and during transport. By «stable», in the meaning of the present invention it is meant that there is no creaming or sedimentation of the droplets of the dispersed fatty phase in the continuous phase, no opacification of the continuous aqueous phase, no aggregating of the droplets, and in particular no coalescence or Ostwald ripening of the droplets, no leakage of materials of the dispersed fatty phase towards the continuous phase, or conversely, and no diffusion and/or sedimentation of the fatty phase pigments.

By «macroscopic» in the meaning of the invention, it is meant to designate a dispersion of which some or all the droplets in the dispersed fatty phase are visible to the naked eye, and preferably a dispersion in which the droplets having a diameter greater than or equal to 150 μm represent a volume greater than or equal to 60%, even greater than or equal to 70%, preferably greater than or equal to 80%, and better still greater than or equal to 90% of the total volume of the dispersed phase, and/or at least 60%, even at least 70%, preferably at least 80%, and better still at least 90%, of the droplets have a mean diameter greater than or equal to 150 μm.

Preferably, the aforementioned diameter is greater than or equal to 250 μm, in particular greater than or equal to 500 μm, even greater than or equal to 1 000 μm, and better still between 150 μm and 3000 μm, preferably between 250 μm and 2000 μm, and in particular between 500 μm and 1 500 μm.

The droplets of a dispersion of the invention are advantageously substantially spherical and/or advantageously have apparent monodispersity (i.e. they are perceived by the eye as being spheres of identical diameter). In the remainder of the present description, the droplets can indifferently be designated by the terms «macroscopic droplets» or «droplets (G1)».

Therefore, in the dispersion of the invention, the constituent phases thereof form a macroscopically non-homogeneous mixture.

The inventors have observed that an increase in the mean diameter of the droplets G1 is correlated with an improvement in the aforementioned advantages of a dispersion according to the invention.

In the present invention, the above-mentioned dispersions can indifferently be designated by the term “emulsions”.

By «immiscible» or «non-miscible» in the meaning of the present invention, it is meant to designate that the solubility of a first phase in a second phase is advantageously lower than 5% by weight.

In one embodiment, a dispersion of the invention does not comprise a surfactant.

The invention also concerns a composition comprising at least one dispersion such as defined above

Unless otherwise stated, in the entire description, temperature is considered to be ambient temperature (e.g. T=25° C.±2° C.) and pressure is atmospheric pressure (760 mm de Hg, i.e. 1.013.105 Pa or 1 013 mbar).

### Viscosity

The viscosity of a dispersion, even of a composition of the invention, can vary to a large extent which allows the obtaining of varied textures.

In one embodiment, a dispersion of the invention has viscosity of 1 mPa·s to 500 000 mPa·s, preferably from 10 mPa·s to 300 000 mPa·s, better still from 400 mPa·s to 100 000 mPa·s, and more particularly from 1 000 mPa·s to 30 000 mPa·s, such as measured at 25° C.

Viscosity is measured at ambient temperature, e.g. T=25° C.±2° C. and at ambient pressure e.g. 1013 mbar, with the method described in WO2017046305.

### Dispersion

A dispersion of the invention is liquid at ambient temperature and ambient pressure. In other words, a dispersion of the invention is not in solid form, particularly not in compact, powder or stick form.

The droplets G1 of a dispersion of the invention can be single-phase or multiphase. Therefore, the droplets comprise a core (comprising at least one fatty phase) and a shell (or membrane or skin) fully encapsulating the core. The core is preferably liquid at 25° C. The core itself may comprise one or more phases. In general, the at least one pigment is included in the phase (or one of the phases) forming the core.

In one embodiment, the droplets of a dispersion of the invention comprise a core that is liquid or at least partly gelled or a least partly thixotropic, and a shell fully encapsulating said core, said core being single-phase and in particular based on a fatty (or oily) phase. Said type of droplet leads to a simple dispersion having two separate phases, an inner liquid phase, or at least partly gelled or at least partly thixotropic, formed by at least the fatty phase, and an outer aqueous phase preferably in the gelled state surrounding the inner phase.

In another particular embodiment the droplets of a dispersion of the invention comprise a core that is liquid or at least partly gelled or at least partly thixotropic, and a shell fully encapsulating said core, said core comprising an intermediate droplet of an intermediate phase and at least one and preferably a single inner droplet of an inner phase arranged in the intermediate droplet, at least one of the intermediate and/or inner phase(s) forming the fatty phase, and the pigment(s) being included in the intermediate phase and/or inner phase.

Advantageously, the intermediate phase is oily and the inner phase is aqueous or formed of a different oily phase and non-miscible at ambient temperature and atmospheric pressure with said intermediate phase. Said type of droplet leads to a complex dispersion meaning that the liquid, viscous or thixotropic core comprises a single intermediate droplet of an intermediate phase, and at least one and preferably a single inner droplet of an inner phase arranged in the intermediate droplet.

In one variant, the core comprises a continuous intermediate phase within which there are a plurality of droplets of inner phase(s).

In one particular embodiment:
the continuous aqueous phase can be in the form of a direct emulsion (oil-in-water), said emulsion comprising a continuous aqueous phase and a dispersed fatty phase in the form of droplets (G2), the size of the droplets (G2) preferably being smaller than the size of droplets (G1);
and/or
the fatty phase, even the intermediate phase and/or inner phase for a complex dispersion such as defined above, can be in the form of an invert emulsion (water-in-oil), said emulsion comprising a continuous fatty phase and a dispersed aqueous phase in the form of droplets (G3), the size of droplets (G3) necessarily being smaller than the size of droplets (G1) and preferably microscopic.

In particular, the size of droplets (G2) and/or (G3) is smaller than 500 μm, preferably smaller than 400 μm, in particular smaller than 250 μm, better still smaller than 150 μm, in particular smaller than 100 μm, even smaller than 20 μm, and better still smaller than 10 μm. Preferably, the size of droplets (G2) and/or (G3) is between 0.1 μm and 200 μm, preferably between 0.25 μm and 100 μm, in particular between 0.5 μm and 50 μm, preferably between 1 μm and 20 μm, and better still between 1 μm and 10 μm, even between 3 μm and 5 μm.

Optionally, the droplets (G2) and/or (G3) comprise a shell formed of at least one anionic polymer, in particular a carbomer, and of at least one cationic polymer, in particular an amodimethicone, said anionic and cationic polymers being such as defined below.

Advantageously, the droplets (G2) and/or (G3) are not macroscopic, and are therefore microscopic i.e. not visible to the naked eye.

In other words, the droplets (G2) and/or (G3) are different and independent of droplets (G1).

These droplets (G2) and/or (G3) of reduced size allow the providing of an effect on texture. A dispersion of the invention comprising said finely dispersed droplets (G2) and/or (G3) displays improved qualities of smoothness.

Advantageously, the droplets (G2) and/or (G3) may additionally comprise at least one pigment, the same or differing from the pigment(s) contained in the fatty phase of droplets (G1).

Advantageously, the intermediate phase also comprises at least one lipophilic gelling agent, in particular as defined below. The gelling agent in particular contributes towards improving the suspending of the inner droplet(s) arranged in the intermediate droplet of the droplets of a dispersion of the invention in this embodiment. In other words, the gelling agent allows the preventing/avoiding of phenomena of creaming or sedimentation of the inner droplet(s) arranged in the intermediate droplet of the droplets (G1) of a dispersion of the invention, in this embodiment.

### Continuous Aqueous Phase

In one embodiment, the aqueous phase has viscosity of between 400 mPa·s and 100 000 mPa·s, preferably between 800 mPa·s and 30 000 mPa·s, such as measured at 25° C.

This viscosity is measured following the method described above.

The continuous phase of the dispersions comprises water. In addition to distilled or deionized water, one water suitable for the invention can also be a spring water or floral water.

In one embodiment, the weight percentage of water in the continuous aqueous phase is at least 30%, preferably at least 40%, in particular at least 50%, and better sill at least 60%, in particular between 70% and 98%, and preferably between 75% and 95%, relative to the total weight of said continuous phase.

The continuous aqueous phase of the dispersion of the invention may further comprise at least one base. It may comprise a single base or a mixture of several different bases. The presence of at least one base in said continuous aqueous phase contributes in particular towards raising the viscosity thereof.

In one embodiment, the base included in the aqueous phase is a mineral base.

In one embodiment, the mineral base is chosen from the group formed by hydroxides of alkali metals and hydroxides of alkaline-earth metals.

Preferably, the mineral base is a hydroxide of alkali metals and in particular NaOH.

In one embodiment, the base included in the aqueous phase is an organic base. Among organic bases, mention can be made for example of ammonia, pyridine, triethanolamine, aminomethylpropanol, or triethylamine.

A dispersion of the invention may comprise from 0.01% to 10% by weight, preferably from 0.01% to 5% by weight, more preferably from 0.02% to 1% by weight of base, preferably a mineral base and in particular NaOH, relative to the total weight of said dispersion.

Preferably, the continuous aqueous phase, even the dispersion of the invention, does not comprise a surfactant.

### Shell of the Droplets

The droplets (G1) of the dispersed fatty phase comprise a shell comprising at least one anionic polymer and at least one cationic polymer.

In the invention, the droplets obtained can have a very thin shell, in particular having a thickness of less than 1% the diameter of the droplets.

The thickness of the shell is therefore preferably less than 1 μm and therefore too small to be measured with optical methods.

In one embodiment, the thickness of the shell of the droplets is less than 1 000 nm, in particular less than 1 to 500 nm, preferably less than 100 nm, advantageously less than 50 nm, most preferably less than 10 nm.

Measurement of the thickness of the shell of the droplets of the invention can be carried out by Small-Angle X-ray Scattering, such as implemented by Sato et al. J. Chem. Phys. 111, 1393-1401 (2007).

For this measurement, the droplets are produced using deuterated water, then washed three times with a deuterated oil e.g. a deuterated oil of hydrocarbon type (octane, dodecane, hexadecane).

After washing, the droplets are transferred to a Neutron cell to determine the spectrum I (q); q being the wavelength vector.

On this spectrum, conventional analytical processing is performed (REF) to determine the thickness of the hydrogenated (non-deuterated) shell.

Therefore, no resistance connected with rupture of the shell is felt by the user at the time of application onto a keratin material, and no residual deposit of said shell is found. The term evanescent shell is used.

The droplets of a dispersion of the invention, through the type and properties of their shell, therefore differ from solid capsules i.e. from capsules having a solid membrane such as those for example described in WO2010/063937.

The shell surrounding the droplets of the dispersed phase particularly imparts sufficient strength to the droplets and therefore reduces and even prevents coalescence thereof.

This shell is typically formed by coacervation, i.e. by precipitation of polymers having opposite charges. Within a coacervate, the bonds linking together the charged polymers are of ionic type, and are generally stronger than the bonds contained in a membrane of surfactant type.

The shell is formed by coacervation of at least two polymers having opposite charges (or polyelectrolyte) and preferably in the presence of a first polymer of cationic type and a second polymer differing from the first polymer and of anionic type. These two polymers act as rigidifying agents for the membrane.

The formation of the coacervate between these two polymers can be obtained by modifying the conditions of the reaction medium (temperature, pH, reagent concentrations, etc.).

The coacervation reaction results from neutralization of these two polymers of opposite polarity and allows the formation of a membrane structure via electrostatic interactions between the anionic polymer and cationic polymer. The membrane thus formed around each droplet typically forms a shell which fully encapsulates the core of the droplet, and therefore isolates the core of the droplet from the continuous aqueous phase.

Advantageously, one of the first and second charged polymers is a lipophilic polymer able to be ionized in contact with an aqueous phase, the other of the first and second charged polymers is a hydrophilic polymer able to be ionized.

### Anionic Polymer

In the present description, by “polymer of anionic type” or «anionic polymer» it is meant a polymer comprising chemical functions of anionic type. The term anionic polyelectrolyte can also be used.

By “chemical function of anionic type”, it is meant a chemical function AH capable of yielding a proton to give a function A. Depending on the conditions of the medium in which it is included, a polymer of anionic type therefore comprises chemical functions in AH form, or else in the form of its conjugate base A.

As examples of chemical functions of anionic type, mention can be made of carboxylic acid functions —COOH, optionally present in the form of a carboxylate anion —COO⁻.

As examples of polymers of anionic type, mention can be made of any polymer formed by polymerization of monomers of which at least one portion carries chemical functions of anionic type, such as carboxylic acid functions. Said monomers are for example acrylic acid, maleic aid, or any ethylenically unsaturated monomer comprising at least one carboxylic acid function. For example, it may be an anionic polymer comprising monomers units having at least one chemical function of carboxylic acid type.

Preferably, the anionic polymer is hydrophilic i.e. soluble or dispersible in water.

Among examples of polymers of anionic type suitable for implementing the invention, mention can be made of copolymers of acrylic acid or maleic acid and other monomers such as acrylamide, alkyl acrylates, C₅-C₈ alkyl acrylates, C₁₀-C₃₀ alkyl acrylates, C₁₂-C₂₂ alkyl methacrylates, methoxypolyethyleneglycol methacrylates, hydroxyester acrylates, crosspolymer acrylates, and mixtures thereof.

In the invention, a polymer of anionic type is preferably a carbomer such as described below. This polymer can also be an acrylates/C₁₀₋₃₀ alkyl acrylate Crosspolymer (INCI name).

In one embodiment, the shell of the droplets comprises at least one anionic polymer e.g. a carbomer.

In the invention and unless otherwise stated, by “carbomer” it is meant an optionally crosslinked homopolymer derived from polymerization of acrylic acid. It is therefore a poly (acrylic acid), optionally crosslinked. Among the carbomers of the invention, mention can be made of those marketed under the trade names Tego®Carbomer 340FD by Evonik, Carbopol® 981 by Lubrizol, Carbopol ETD 2050 by Lubrizol, or Carbopol Ultrez 10 by Lubrizol.

In one embodiment, by “carbomer” or “Carbopol®”, it is meant a high molecular weight acrylic acid polymer crosslinked with allyl sucrose or allyl ethers of pentaerythritol (Handbook of Pharmaceutical Excipients, 5^{eme} Edition, pIII). For example, it is Carbopol®910, Carbopol®934, Carbopol®934P, Carbopol®940, Carbopol®941, Carbopol®71G, Carbopol®980, Carbopol®971P or Carbopol®974P. In one embodiment, the viscosity of said carbomer is between 4 000 and 60 000 cP at 0.5% w/w.

Carbomers have other names: polyacrylic acids, carboxvinyl polymers or carboxy polyethylenes.

A dispersion of the invention may comprise from 0.01% to 5% by weight, preferably from 0.05% to 2%, and more preferably from 0.10% to 0.5% of anionic polymer(s), carbomer(s) in particular, relative to the total weight of said dispersion.

In the invention, the dispersions of the invention may comprise a carbomer and an acrylates/C₁₀₋₃₀ alkyl acrylate Crosspolymer.

The aqueous phase of the invention may also comprise at least one crosslinked polymer or at least one crosslinked copolymer, said crosslinked polymer or crosslinked copolymer comprising at least one unit derived from polymerization of one of the following monomers: acrylic or methacrylic acid, alkyl acrylate or methacrylate having 1 to 30 carbon atoms, or the salts thereof.

This is particularly the case when a dispersion of the invention comprises at least one fragrance such as defined below.

The aqueous phase may also comprise a mixture of crosslinked polymers or a mixture of crosslinked copolymers, or a mixture of crosslinked polymers and crosslinked copolymers.

In the invention, by the term “unit derived from polymerization of a monomer” it is meant that the polymer or copolymer is a polymer or copolymer obtained by polymerization or copolymerization of said monomer.

In one embodiment, the crosslinked polymer or crosslinked copolymer is a crosslinked polyacrylate.

The crosslinked copolymers and polymers of the invention are anionic.

In one embodiment, the copolymer is a copolymer of unsaturated carboxylic acid and unsaturated carboxylate of C₁₋₃ alkyl, preferably C₁-C₄. Said copolymer comprises at least one hydrophilic unit of the type unsaturated olefinic carboxylic acid and at least one hydrophobic unit of the type (C₁-C₃₀)alkyl ester of unsaturated carboxylic acid.

Preferably, these copolymers are chosen from among those having a hydrophilic unit of the type unsaturated olefinic carboxylic acid corresponding to the monomer of following formula (I)

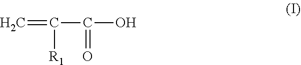

where: R₁ is H or CH₃ or C₂H₅, i.e. units of acrylic acid, methacrylic acid or ethylacrylic acid in which the hydrophobic unit of the type (C₁-C₃₀)alkyl ester of unsaturated carboxylic acid corresponds to the monomer of following formula (II):

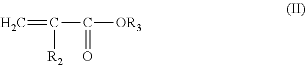

where: R₂ is H or CH₃ or C₂H₅ (i.e. acrylate, methacrylate or ethyl acrylate units) and preferably H (acrylate units) or CH₃ (methacrylate units), R₃ designating a C₁-C₃₀ alky radical, and preferably C₁-C₄.

Among these types of copolymers, more particular use is made of those formed from a mixture of monomers comprising:
(i) essentially acrylic acid,
(ii) an ester of formula (II) described above and where R₂ is H or CH₃, R₃ designating an alkyl radical having 1 to 4 carbon atoms,
(iii) and a crosslinking agent which is a well-known copolymerizable unsaturated polyethylene monomer such as diallyl phthalate, trimethylolpropane tri(meth) acrylate, diallyl itaconate, diallyl fumarate, diallyl maleate, zinc(meth) acrylate, allyl(meth) acrylate, divinylbenzene, (poly)ethyleneglycol dimethacrylate, methylene-bis-acrylamide, and castor oil.

In one embodiment, the crosslinked polymer or crosslinked copolymer is a polymer or copolymer of acrylic acid and/or methacrylic acid and/or alkyl acrylate having 1 to 30 carbon atoms, preferably 1 to 4 carbon atoms, and/or alkyl methacrylate having 1 to 30 carbon atoms, preferably 1 to 4 carbon atoms.

In one embodiment, the crosslinked copolymer is a crosslinked copolymer of methacrylic acid and alkyl acrylate having 1 to 4 carbon atoms, preferably 2 carbon atoms.

In the invention, unless otherwise stated, by «crosslinked copolymer of methacrylic acid and alkyl acrylate having 1 to 4 carbon atoms», it is meant a crosslinked copolymer resulting from polymerization of a monomer of methacrylic acid and a monomer of alkyl acrylate having 1 to 4 carbon atoms.

Preferably, in this copolymer, the methacrylic acid represents from 20% to 80% by weight, preferably from 35% to 65% by weight of the total weight of the copolymer.

Preferably, in this copolymer the alkyl acrylate represents from 15% to 80% by weight, preferably from 35% to 65% by weight of the total weight of the copolymer.

In particular, the alkyl acrylate is chosen from among alkyl methacrylate, ethyl acrylate and butyl acrylate.

In one embodiment, the crosslinked polymer or crosslinked copolymer of the invention, contained in the continuous aqueous phase, is chosen from the group composed of the following polymers or copolymers: Acrylates Copolymer, Acrylates crosspolymer-4, Acrylates crosspolymer-3, Polyacrylate-2 Crosspolymer and Polyacrylate-14 (INCI names).

Among said above polymers, particular preference is given in the invention to the products sold by LUBRIZOL under the trade names Fixate Superhold (INCI name=Polyacrylate-2 Crosspolymer), Fixate Freestyle Polymer (INCI name=Acrylates crosspolymer-3), Carbopol® Aqua SF1 (INCI name=Acrylates copolymer) and Carbopol® Aqua SF2 (INCI name=Acrylates crosspolymer-4).

Preferably, the crosslinked copolymer is Carbopol® Aqua SF1 (INCI name=Acrylates copolymer).

In one embodiment, the crosslinked copolymer is chosen from among the crosslinked copolymers of acrylic or methacrylic acid and alkyl acrylates having 1 to 4 carbon atoms.

In the invention, the dispersion of the invention may comprise from 0.1% to 10% by weight, preferably from 0.5% to 8% by weight, and more preferably from 1% to 3% by weight of crosslinked polymer(s) or crosslinked copolymer(s) relative to the total weight of said dispersion.

In the invention, the dispersions of the invention may comprise a carbomer and a crosslinked copolymer Carbopol® Aqua SF1 (INCI name=Acrylates copolymer).

### Cationic Polymer

The droplets, and in particular the shell of said droplets also comprise at least one polymer of cationic type. They may also comprise several polymers of cationic type. This cationic polymer is the one mentioned above which forms the shell by coacervation with the anionic polymer.

In the present application, unless otherwise stated, by “polymer of cationic type” or «cationic polymer» it is meant a polymer comprising chemical functions of cationic type. The term cationic polyelectrolyte can also be used.

Preferably, the cationic polymer is lipophilic or liposoluble.

In the present application and unless otherwise stated, by “chemical function of cationic type”, it is meant a chemical function B capable of capturing a proton to give a function BH⁺. Depending on the conditions of the medium in which it is contained, the polymer of cationic type therefore comprises chemical functions in form B or else in form BH⁺, the conjugate acid thereof.

As examples of chemical functions of cationic type, mention can be made of primary, secondary, and tertiary amines optionally present in the form of ammonium cations.

As examples of polymers of cationic type, mention can be made of any polymer formed by polymerization of monomers of which at least some carry chemical functions of cationic type, such as primary, secondary, or tertiary amine functions.

Said monomers are aziridine for example, or any ethylenically unsaturated monomer comprising at least one primary, secondary, or tertiary amine function.

Among the examples of cationic polymers suitable for implementing the invention, amodimethicone can be cited, a derivative of a silicone polymer (polydimethylsiloxane, also called dimethicone), modified by primary amine and secondary amine functions.

Mention can also be made of derivatives of amodimethicone, for example copolymers of amodimethicone, aminopropyl dimethicone, and more generally linear or branched silicone polymers comprising amine functions.

The copolymers of bis-isobutyl PEG-14/amodimethicone, Bis(C13-15 Alkoxy) PG-Amodimethicone, Bis-Cetearyl Amodimethicone and bis-hydroxy/methoxy amodimethicone can be cited.

Polymers of polysaccharide type comprising amine functions can also be cited, such as chitosan or derivatives of guar gum (guar hydroxypropyltrimonium chloride).

Polymers of polypeptide type comprising amine functions can also be cited, such as polylysine.

Mention can also be made of polymers of polyethyleneimine type comprising amine functions, such as linear or branched polyethyleneimine.

In one embodiment, the droplets and in particular the shell of said droplets comprise a cationic polymer which is a silicone polymer modified by a primary secondary or tertiary amine function, such as amodimethicone.

In one embodiment, the droplets and in particular the shell of said droplets comprise amodimethicone.

In one particular preferred embodiment, the cationic polymer meets the following formula:

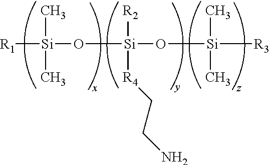

where:
R₁, R₂ and R₃, are each independently OH or CH₃;
R₄ is a group —CH₂— or group —X—NH— where X is a C₃ or C₄ divalent alkylene radical;
x is an integer between 10 and 5 000, preferably between 30 and 1 000, and better still between 80 and 300;
y is an integer between 1 and 1 000, preferably between 2 and 1 000, and better still between 4 and 100, better still between 5 and 20; and
z is an integer between 0 and 10, preferably between 0 and 1, and better still it is 1.

In the above-mentioned formula, when R₄ represents a group —X—NH—, X is bonded to the silicon atom.

In the above-mentioned formula, R₁, R₂ and R₅ are preferably CH₃.

In the above-mentioned formula, R₄ is preferably a group —(CH₂)₃—NH—.

In one particularly preferred embodiment, the cationic polymer meets following formula (1-1):

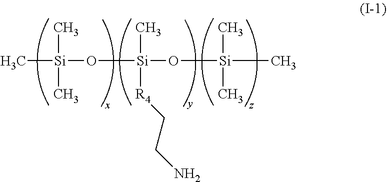

where:
R₄ is such as previously defined, and is preferably a group —(CH₂)₃—NH—;
x is an integer between 80 and 300, de preferably between 100 and 200;
y is an integer between 5 and 20, preferably between 5 and 15; and
z is an integer between 0 and 1, preferably it is 1.

The cationic polymer of the invention can be one of the following commercial products: CAS 3131 by Nusil, KF 8005 S or KF 8004 by Shin Etsu, Silsoft AX or SF 1708 by Momentive, and DC 8500, DC 2-2078 or DC 2-8566 by Dow Corning.

The cationic polymer of the invention can be an amodimethicone such as one of the following commercial products: CAS 3131 by Nusil, KF 8005 S or KF 8004 by Shin Etsu, SF 1708 by Momentive, and DC 2-8566 by Dow Corning.

In one preferred embodiment, the quantity of amine functions contributed by the cationic polymer in the fatty phase is between 14.4 μmol and 28.8 μmol, preferably between 18 μmol and 25.2 μmol, in particular between 20 μmol and 23 μmol, and better still between 21 μmol and 22 μmol, per gram of fatty phase.

In the invention, a dispersion may comprise from 0.75% to 2.25%, preferably 1% to 2%, in particular 1.25% to 1.75%, and better still 1.35% to 1.60% by weight of cationic polymer(s) in particular amodimethicone(s), relative to the total weight of the fatty phase.

These contents are advantageous since they further improve the properties of a dispersion of the invention, in particular in terms of sphericity and mechanical strength of the droplets (G1).

In the invention, and unless otherwise stated, by «quantity of amine functions contained in (or contributed by) the cationic polymer», it is meant the quantity of amines carried by the cationic polymer.

For obvious reasons, the amine functions under consideration contributed by the cationic polymer are those able to react with the anionic polymer, in particular with the carboxylic groups (or functions) carried by said anionic polymer. They are preferably amine functions present on the branched chains of the cationic polymer.

Therefore, advantageously, at least 50%, preferably at least 60%, in particular at least 70%, better still at least 80%, preferably at least 90%, and more particularly at least 99% of the amine functions carried by the cationic polymer are able to react with the anionic polymer, in particular with the carboxylic groups carried by said anionic polymer.

To ensure efficient bridging between the amine functions of the cationic polymer and the carboxylic function(s) of the anionic polymer, as indicated previously, a cationic polymer of the invention comprises at least two amine functions. In this respect and preferably, a cationic polymer of the invention comprises at least two amine functions located on different branched chains of said cationic polymer. In other words, a cationic polymer of the invention comprises at least two branched chains, the same or different, each branched chain comprising at least one amine function able to react with the anionic polymer, in particular with at least one carboxylic group carried by said anionic polymer.

### Method for Determining the Quantity of Amine Functions Contained in the Cationic Polymer, in Particular Amodimethicone

The quantity of amine functions contained in the cationic polymer can be measured in particular with the following method:

### Parameters

Unless otherwise stated, the test described below is conducted under ambient conditions, namely ambient temperature (25° C.) and at 30%-70% relative humidity.

Tris(hydroxymethyl) aminomethane (TRIS) (CAS No 77-86-1) is dried at 110° C. for at least 2 hours, and cooled over a week.

### Method

Preparation of the Solvent Solution (i.e. Solution A)
Measure 1000 ml of toluene of HPLC quality in an amber-coloured bottle.
Add 10 ml of distilled water.
Add 990 ml of isopropyl alcohol.
Close the bottle and gently mix the solution for about one minute.
Solution A is obtained.

Check that solution A is slightly acid (pH ˜ 5) by adding 3 drops of Bromocresol Purple indicator solution to an aliquot of 100 ml of solution A.

Shake for about 30 seconds.

The solvent solution should appear yellow/green after mixing.

Preparation of 0.5% of Bromocresol Green Indicator Solution (i.e. Solution B)
In a bottle, measure 0.50±0.01 grams of Bromocresol Green dye.
Add 100 ml of ethanol.
Close the bottle and shake vigorously.
Solution B is obtained.
   Titration of HCl Solution (i.e. Solution C)

Measure a suitable amount in TRIS. Record the mass of TRIS M1.

NOTE: with 0.1 N HCl, add about 0.2 grams of dried TRIS; with 1 N HCl, add about 1.8 grams of dried TRIS.

Add 100 ml of demineralized water.

Add 3 drops of solution B.

Agitate for about 3 minutes.

N.B: the TRIS solution should appear blue after agitation.

Set up a 25 ml burette.

Fill the burette with standard HCl solution. Purge air from the top of the burette allowing about 1.0 ml of HCl to flow through the top of the burette.

Reading of the standard HCl standard solution in the burette should be between 0.00 ˜1.00 ml.

Record the volume of HCl in the burette V1.

Start titration with the addition of the standard HCl solution by adding 0.5 ml to the TRIS solution whilst stirring said TRIS solution with a magnetic stirrer.

Take care when approaching the final titration point by slowly adding small drops of standard HCl solution to the TRIS solution until the colour of the TRIS solution changes from blue to yellow and remains yellow for at least 60 seconds.

NOTE: The colour of the TRIS solution may temporarily change from blue to green close to the point where the titrant is added. As titration product, the change in colour of the TRIS solution will become more dispersed and will last longer. Titration is completed when the colour of the TRIS solution changes to yellow with the addition of the last drop of reagent and the change in colour lasts for at least 60 seconds.

Record the final volume of HCl in the burette V2.

Repeat until three successful titrations have been completed.

### Titration of the Cationic Polymer

Measure 0.05 to 5 grams of the cationic polymer under consideration, amodimethicone in particular, according to the amine content in said cationic polymer.

Record the mass of cationic polymer M2.

Add 50 ml of solution A.

NOTE: If the cationic polymer does not dissolve in solution A, first add the isopropanol, then the toluene and water in a suitable ratio.

Add 3 drops of solution B.

Solution D is obtained.

Mix solution D for at least 5 minutes or until solution D appears homogeneous.

N.B.: Solution D should change from blue to green after agitation.

Set up a 25 ml burette

Fill the burette with standard HCl solution. Purge air from the top of the burette allowing about 1.0 ml of HCl to flow through the top of the burette.

Reading of the standard HCl solution in the burette should be between 0.00˜1.00 ml.

Record the volume of standard HCl solution in the burette V3.

Start titration by adding the standard HCl solution to above solution D in additions of 0.5 ml whilst stirring solution D with a magnetic stirrer.

Take care when approaching the final point of titration by slowly adding small drops of standard HCl solution until the colour of solution D changes from blue to yellow and remains yellow for at least 60 seconds.

NOTE: The colour of solution D will temporarily change from blue to yellow or green close to the point where the titrant is added. In some samples, the colour may change from blue to purple to yellow. Titration is completed when the colour of solution D changes to yellow by adding the last drop of reagent and the change in colour lasts for at least 60 seconds.

Record the final volume of standard HCl solution in the burette: V4.

### Calculations

Calculation of the normality of the standard hydrochloric acid solution:

$$N = \frac{{M}_{1}}{( 0.12114 ) ( {V}_{2} - {V}_{1} )}$$

N=normality of HCl, in N
M1=mass of TRIS, in grams
V1=initial volume of standard HCl solution in the burette, in mL
V2=final volume of standard HCl solution in the burette, in mL

Calculation of the content of amine groups (in millimoles of amine/grams of cationic polymer)

$$Amine = \frac{N \times ( {V}_{4} - {V}_{3} )}{{M}_{2}}$$

M2=mass of cationic polymer, in grams
V3=initial volume of standard HCl solution in the burette, in mL
V4=final volume of standard HCl solution in the burette, in mL
N=indicates the normality of standard HCl solution in the burette, in N

### Formulas

Calculation of normality of the standard HCl solution (N):

$$= ( gram TRIS ) ( \frac{1 mole TRIS}{121.135 g TRIS} ) \times ( \frac{1 eq TRIS}{1 mole TRIS} ) \times ( \frac{1 eq HCl}{1 eq TRIS} ) \times ( \frac{1}{mL HCl} ) \times ( \frac{1000 mL}{1 L} )$$

$$N ( eq / L )$$

Calculation of the amine content (in millimoles/grams of cationic polymer):

$$= ( mL de HCl ) \times ( \frac{1 eq {d}^{′} HCl}{L {d}^{′} HCl} ) \times ( \frac{1}{gram} ) \times ( \frac{1 mole Amine}{1 eq {d}^{′} HCl} ) \times ( \frac{1 L HCl}{10 \; 00 mL HCl} ) \times ( \frac{1 \; 000 mmole Amine}{1 mole amine} )$$

$$Amine millimole / gram$$

### Method for Determining the Quantity of Amine Functions Contributed by the Cationic Polymer, in Particular Amodimethicone, in the Fatty Phase

Starting from a specific cationic polymer, it is within the reach of persons skilled in the art to carry out suitable calculations to determine the required quantity of cationic polymer under consideration to meet requirements in terms of quantity of amine functions contributed by the cationic polymer in the fatty phase such as targeted in the present invention.

In this respect, the following calculation method can be cited:

$$Q = A \times T$$

where:
Q is the quantity of amine functions contributed by the cationic polymer in the fatty phase (in μmol/g),
A is the mass percent of cationic polymer under consideration in the fatty phase, and
T is the quantity of amine functions carried by said cationic polymer under consideration (in μmol/g of cationic polymer), in particular obtained with the above-described method.

| | Quantity of amine functions |
|---|---|
| Amodimethicone | (in mmol per gram of amodimethicone) |
| CAS-3131 by NUSIL (INCI: | 1.44 |
| Amodimethicone) | |

### Fatty Phase

In the invention, a dispersion comprises a dispersed phase in the form of droplets which, in addition to at least one cationic polymer such as described above, comprises at least one fatty (or oily) phase, at least one pigment and at least one cationic polymer such as previously described and optionally at least one lipophilic gelling agent differing from the anionic and cationic polymers described above.

### Oils

The oily phase may comprise at least one oil, preferably in which the cationic polymer such as described above is soluble.

By «oil» it is meant a fat, liquid at ambient temperature.

Among oils able to be used in the composition of the invention, mention can be made of:
hydrocarbon oils of vegetable origin, such as hydrogenated jojoba oil, hydrogenated sunflower seed oil, hydrogenated castor oil, hydrogenated copra oil;
hydrocarbon oils of animal origin such as perhydrosqualene and squalane;
esters and synthetic ethers, in particular of fatty acids, such as oils of formulas RICOOR₂ and R₁OR₂ where R₁ is the remainder of a C₈ to C₂₉ fatty acid, and R₂ is a branched or unbranched C₃ to C₃₀ hydrocarbon chain, e.g. PurCellin oil, isononyl isononanoate, isodecyl neopentanoate, isopropyl myristate, ethyl-2-hexyl palmitate, octyl-2-dodecyl stearate, octyl-2-dodecyl erucate, isostearyl isostearate; hydroxylated esters such as isostearyl lactate, octyl hydroxystearate, octyldodecyl hydroxystearate, diisostearyl malate, triisocetyl citrate, heptanoates, octanoates, fatty alcohol decanoates; polyol, esters such as propylene glycol dioctanoate, neopentylglycol diheptanoate and diethyleneglycol diisononanoate; and the esters of pentaerythritol such as pentaerythrityl tetrabehenate (DUB PTB) or pentaerythrityl tetraisostearate (Prisorine 3631);
linear or branched hydrocarbons of mineral or synthetic origin such as paraffin oils whether or not volatile, and derivatives thereof, Vaseline, polydecenes, hydrogenated polyisobutene such as Parleam oil;
silicone oils e.g. polydimethylsiloxanes (PDMS) whether or not volatile having a linear or cyclic silicone chain, liquid or pasty at ambient temperature, in particular cyclopolydimethylsiloxanes (cyclomethicones) such as cyclohexasiloxane and cyclopentasiloxane; polydimethylsiloxanes (or dimethicones) comprising alkyl, alkoxy or phenyl groups, either pendant or at the end of the silicone chain, groups having 2 to 24 carbon atoms; phenyl silicones such as phenyltrimethicones, phenyldimethicones, phenyltrimethylsiloxydiphenyl-siloxanes, diphenyl-dimethicones, diphenylmethyldiphenyl trisiloxanes, 2-phenylethyltrimethyl-siloxysilicates, and polymethylphenylsiloxanes;
fatty alcohols having 8 to 26 carbon atoms such as cetyl alcohol, stearyl alcohol and mixture thereof (cetylstearyl alcohol), or octyldodecanol;
partially hydrocarbonated and/or siliconated fluorinated oils such as those described in document JP-A-2-295912;
and mixtures thereof.

Skilled persons are able to adjust the type and/or content of oil(s), in particular to ensure satisfactory solubilization (or homogenization) of the pigment(s) and, if any, of the cationic polymer(s).

Therefore, the fatty phase advantageously comprises less than 40%, preferably less than 30%, in particular less than 20% and better still less than 10% by weight of hydrocarbon oil(s) of vegetable origin relative to the total weight of the fatty phase.

Advantageously, a dispersion of the invention comprises at least one non-volatile hydrocarbon oil (or oil H₁) containing more than 90%, preferably more than 95% of fatty acids having a chain length greater than or equal to 18 carbon atoms, preferably greater than or equal to 20 carbon atoms.

Preferably, more than 90%, more preferably more than 95% of the fatty acids of the non-volatile hydrocarbon oil have a chain length of between C₁₈ and C₃₆, more preferably between C₂₀ and C₂₈, and better still between C₂₀ and C₂₂.

By «non-volatile», it is meant an oil having a vapour pressure at ambient temperature and atmospheric pressure, which is nonzero and lower than 0.02 mm Hg (2.66 Pa) and better still lower than 10-3 mm Hg (0.13 Pa).

Therefore, as oil H1, mention can be made of jojoba oil, flax oil, Perilla oil, Inca Inchi oil, rosehip oil, rapeseed oil, hemp oil, sweet almond oil, corn oil, apricot oil, castor oil, Meadowfoam oil (INCI: Limnanthes Alba (Meadowfoam) Seed Oil) and mixtures thereof, preferably jojoba oil and/or Meadowfoam oil, and better still Meadowfoam oil.

The use of oils H1, in particular of Meadowfoam oil, in the fatty phase of a dispersion of the invention has advantageous effects in terms of reducing opacification of the continuous aqueous phase and/or adhesion of droplets to walls of packaging and/or aggregation of droplets.

The oily phase advantageously comprises Meadowfoam oil (e.g. 20% by weight relative to the weight of the oily phase). The presence of this oil, when coupled with Estogel M (via EMC30), increases the mechanical strength of the particles (P), this being particularly useful in a bi-phase composition in which the particles (P) undergo much greater stresses than in a conventional composition i.e. in which the viscosity of the continuous aqueous phase is adjusted to obtain stable suspending of said particles (P).

Preferably, as oil(s), a dispersion of the invention comprises Caprylic/Capric triglyceride, hexyl laurate, and mixtures thereof.

Preferably, a dispersion of the invention, and in particular the fatty phase, does not comprise a crystallizable oil having a melting point (T_{M}) lower than 100° C.

A dispersion of the invention may comprise from 1% to 99.25%, in particular between 1% and 90%, preferably from 5% to 80%, better still from 10% to 70%, and in particular 20% to 60% by weight of oil(s) relative to the total weight of the fatty phase.

### Pigments

The fatty phase of a dispersion of the invention comprises at least one pigment. The use of several pigments allows shading of the colour of the droplet fatty phase, and hence of the dispersion, as desired.

By «pigment» it is meant a colouring chemical substance insoluble in the phase in which it is contained. By «insoluble», it is meant that the solubility at 20° C. of the pigment in the phase in which the pigment is contained is less than 1 g/L, in particular less than 0.1 g/L, preferably less than 0.001 g/L.

Each pigment can independently be an organic, inorganic or hybrid organic-inorganic pigment. They are typically inorganic pigments.

The colouring imparted by a dispersion of the invention can be measured for example by spectrocolorimetry and/or spectrophotocolorimetry.

Coverage corresponds to the capability of a composition to «mask the skin»/«hide imperfections».

The coverage of a composition is measured at a finite thickness of 50 μm for compositions liquid at 25° C. to be applied to the lips, in particular liquid lipsticks, liquid lip gloss and liquid lip balms, and at a thickness of 150 μm for eyeshadows, liquid foundations, mascaras and other liquid make-up products not intended to be applied to the lips. The composition is spread over black matt and white matt contrast test cards e.g. of trademark LENETA Form WPI for black matt cards and Leneta IA for white matt cards. Application can be obtained with an automatic applicator. If the composition is non-homogeneous as is the case for a dispersion of the invention, in particular when the droplets (G1) are macroscopic, a mixing step of said dispersion e.g in a Raneri mixer is carried out before the application step (i.e. spread over the cards) and is preferably carried out until it is homogeneous Measurements are taken on the spread compositions. Reflectance spectra are acquired on a MINOLTA 3700-d spectrophotometer (diffuse measuring geometry and viewing angle of D65/10°, specular component excluded, small aperture (CREISS)) on black and white backgrounds. The spectra are expressed in colorimetric coordinates in the CIELab76 space as specified by the Commission Internationale de l'Eclairage (International commission on Illumination) according to recommendation 15:2004. The contrast ratio or coverage is calculated by obtaining the arithmetic mean of Y against a black background, divided by the mean value of Y against a white background, multiplied by 100.

As pigments, particular mention can be made of titanium dioxide, zinc dioxide, zirconium or cerium oxides, and iron or chromium oxides, manganese violet, ultramarine blue, chromium hydrate and ferric blue, and mixtures thereof. The preferred mineral pigments are iron oxides in particular red iron oxide, yellow iron oxide, brown iron oxide, black iron oxide, titanium dioxide and mixtures thereof.

The pigment is preferably an iron oxide in particular red iron oxide, yellow iron oxide, brown iron oxide, black iron oxide and mixtures thereof.

Each pigment can be a treated or non-treated pigment. In the meaning of the application, by «treated pigment» it is meant a pigment having been treated with an additive improving the dispersibility thereof within an oily or aqueous composition, in particular one of the additives defined below. By «non-treated pigment» or «untreated pigment», it is meant a pigment that has not been treated with said additive.

In the light of the foregoing, the droplet fatty phase of a dispersion of the invention comprises a high content of pigment(s).

Nevertheless, the continuous aqueous phase, even the inner aqueous phase for a complex dispersion such as described above, may also comprise at least one pigment.

Preferably when the phase comprising pigments is a fatty (or oily) phase, said phase also comprises hydrostearic acid or polyhydroxystearic acid such as marketed by Phoenix Chemical under the trade name PELEMOL PHS-8, preferably in an amount of between 0.5% and 10%, in particular between 1.5% and 6%, and better still between 2.5% and 4% by weight, relative to the total weight of the phase under consideration.

The presence of said particular compound(s) is advantageous in that:
they allow reducing of the viscosity of a fatty phase comprising at least one pigment, for example a pigment/oil homogenate (60:40), and all the more so for a phase with high pigment content, hence fluidifying the latter making it more easily processable, in particular with regard to fluidic systems such as described below; and
they allow maintaining of droplet size. The inventors have observed that the use of pigment(s) in a dispersed fatty phase of a dispersion of the invention generally leads to a decrease in droplet size compared with the same dispersion devoid of said pigment(s).

Finally, the maintained integrity of a dispersion of the invention in the presence of this or these compounds is unexpected. This or these compounds generally destabilize the shell comprising at least anionic polymer and at least one cationic polymer.

In one first alternative, the pigment used is a non-treated and non-milled pigment (pigment used «as such»).

In a second alternative, the pigment used has previously undergone treatment so that it is more easily dispersible when formulating the pigment, i.e. in particular more easily dispersible in the phase under consideration. This prior treatment entails grinding the pigment and/or pre-treating the pigment with an additive improving the dispersibility thereof before it is formulated in a series of coloured particles.

The use of a ground pigment and/or pretreated pigment via an additive improving the dispersibility thereof:
contributes towards obtaining a liquid, containing a ground and/or pretreated pigment, that has low viscosity,
contributes towards the preparation of a dispersion of which the dispersed fatty phase has a very high pigment content, comprising more than 23.5%, generally more than 25%, in particular more than 30%, even more than 40% by weight of pigment(s) relative to the weight of dispersed fatty phase,
contributes towards reducing, even preventing sedimentation of the pigment(s) in the phase(s) in which they are contained, and/or
contributes towards reducing, even preventing aggregation of the pigments in the phase(s) in which they are contained.

In general, when several pigments are used, they all undergo the same treatment i.e. they are all ground and/or they are all pretreated. It is possible however that some pigments are ground and non-treated, and others are treated and ground or not ground.

In a first embodiment according to the second alternative, the at least one pigment is pretreated with an additive improving the dispersibility of the pigment.

The type of additive improving the dispersibility of the pigment is dependent on the hydrophilic or lipophilic nature of the phase(s) in which this treated pigment is to be contained.

If a dispersion uses several pretreated pigments, these can be pretreated with the same additives or differing from each other.

An additive improving the dispersibility of the pigment within an oily phase is chosen for example from among hydrogenated lecithin, a silicone, wax, amino acid or salt thereof, an amino acid ester or salt thereof, and mixtures thereof. Hydrogenated lecithin comprises phosphate mono- and di-esters comprising fatty chains promoting the dispersibility of the oily phase. The silicone additive can be obtained either from a precursor of silicone such as an alkoxyalkylsilane e.g. triethoxycaprylsilane, or such as a trialkylsiloxysilicate e.g. trimethylsiloxysilicate, or it can be a silicone such as dimethicone or derivative thereof e.g. bis-hydroxyethoxypropyl dimethicone, or it can be obtained from a mixture of silicone and one of the precursors thereof e.g. a mixture of dimethicone and trimethylsiloxysilicate. The silicone additive can be a hybrid treatment, in particular a mixture of isopropyl titanium triisostearate, bis-hydroxyethoxypropyl dimethicone, PEG-2 soyamine and isophorone diisocyanate (IPDI). The wax can be floral rose wax for example. The preferred amino acid is cystine, and the preferred esters of amino acids are sodium cocoyl glutamate, lauroyl arginine or lauroyl lysine.

An additive improving the dispersibility of the pigment within an aqueous phase is chosen in particular from among an additive of following formula (I):

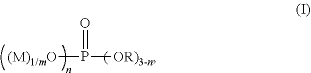

where:
n is 1 or 2,
M is H or a cation,
m is 1 when M is H, and m is the valence of the cation when M is a cation,
R is:
   a group G chosen from among a saccharide or group —[CH₂—CHR₁—O]_{q}—R₂ or —[CH₂—CH(CH₂OH)—O]_{q}—R₂, where:
      q is an integer from 1 to 1000,
      for each unit CH₂—CHR₁—O, R₁ is independently H or a methyl,
      R₂ is H or an alkyl having 1 to 3 carbon atoms, and
   a hydrocarbon chain having 1 to 500 carbon atoms substituted by one or more PHOSPHATE (OF FORMULA OPO₃(M)_{2/M}) AND/OR HYDROXYL (OH) GROUPS G.

The group —[CH₂—CHR₁—O]_{q}—R₂ where R₁ is H corresponds to a polyethylene glycol (PEG). The group —[CH₂—CHR₁—O]_{q}—R₂ where R₁ is a methyl corresponds to a polypropylene glycol (PPG). The group —[CH₂—CH(CH₂OH)—O]_{q}—R′ corresponds to a polyglycerol.

Typically, q is an integer from 1 to 500, in particular from 1 to 100, preferably from 1 to 60.

Preferably, n is 2 and the additive has the following formula (I′):

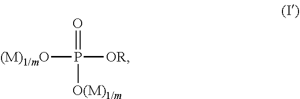

where M, m, and R are such as defined above.

In the meaning of the present application, a hydrocarbon chain has 1 to 500 carbon atoms, in particular 1 to 50, typically 1 to 10 carbon atoms, and preferably 1 to 5 carbon atoms. The hydrocarbon chains can be linear, branched or cyclic. The preferred hydrocarbon chains are the alkyl groups (preferably having 1 to 10 carbon atoms, in particular 1 to 5 carbon atoms, preferably 1 to 3 (such as the methyl, ethyl, n-propyl and isopropyl groups), alkenyl groups (preferably having 2 to 10 carbon atoms, in particular 2 to 6), aryl groups (preferably having 6 to 10 carbon atoms), arylalkyl groups (preferably having 7 to 10 carbon atoms) or alkylaryl groups (preferably having 7 to 10 carbon atoms). The vinyl group is the preferred alkenyl group. The phenyl group is the preferred aryl group.

A saccharide can be a mono- or polysaccharide. The preferred saccharides are mono- or disaccharide, in particular monosaccharides such as glucose, galactose or fructose.

M can particularly be an inorganic cation such as Ag³⁺, Al³⁺, Fe³⁺, Fe^{2+,} Ag²⁺, Zn²⁺, Sn²⁺, Ca²⁺, Ba²⁺, Ag⁺, Na⁺, or an organic cation such as a diethanolammonium (DEA) (H₃N⁺—(CH₂)₂—OH) or a quaternary ammonium.

The following additives of following formulas (II), (III) or (IV) are particularly suitable for implementing the invention:

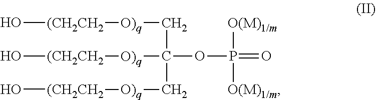

where M, m, and q are such as defined above,

(which corresponds to an additive of formula (I) where n is 2 and R is an isopropyl hydrocarbon chain each of the carbon atoms thereof being substituted by a group G which represents —[CH₂—CHR₁—O]_{q}—R₂ where R₁ and R₂ are H),

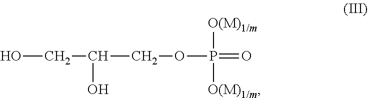

where M and m are such as defined above,

(which corresponds to an additive of formula (I) where n is 2, R is a group G of formula —[CH₂—CH(CH₂OH)—O]_{q}—R₂ where q is 1 and R₂ is H),

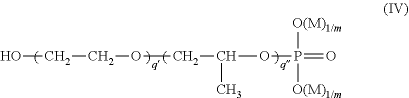

where M and m are such as defined above and q′ and q″ are each independently an integer of 0 to 1000, generally from 0 to 500, in particular from 0 to 100, preferably from 0 to 60, such that the sum of q′ and q″ is independently an integer of 1 to 1000, (which corresponds to an additive of formula (I) where n is 2, R is a group G of formula —[CH₂—CHR₁—O]_{q}—R₂ where q is the sum of q′ and q″ and, for the q″ first units R₁ is a methyl, and for the q′ last units, R₁ is H, and R₂ is H).

The additives of following formulas (V) and (VI) are also suitable:

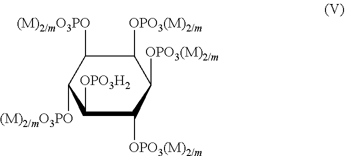

(which corresponds to an additive of formula (I) where n is 2, R is a cyclohexyl hydrocarbon chain substituted at positions 2, 3, 4, 5 and 6 by a phosphate group of formula OPO₃H₂),

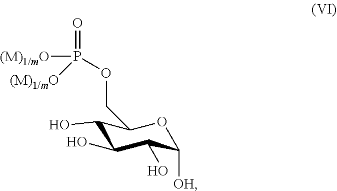

(which corresponds to an additive of formula (I) where n is 2, R is a methyl hydrocarbon chain bonded to a glucose group G).
where M and m are such as defined above.

The following additives are particularly preferred:
glycereth-26 phosphate, of following formula (II′):

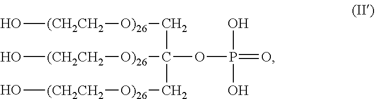

(which corresponds to an additive of formula (II) where M is H and m is 1), this additive advantageously being commercially available e.g. from Croda®,
glycerophosphate, of following formula (III′):

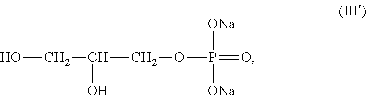

(which corresponds to an additive of formula (III) where M is Na and m is 1), this additive advantageously being commercially available e.g. from Dr Paul Lohman®,
PEG-26 PPG-30 diethanolammonium phosphate of following formula (IV′):

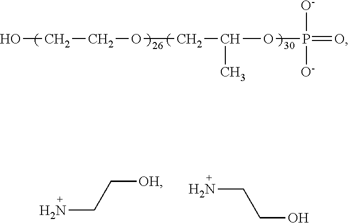

(which corresponds to an additive of formula (IV) where M is a diethanolammonium cation and m is 1), this additive advantageously being commercially available e.g. from Innospec®,
phytic acid of following formula (V′):

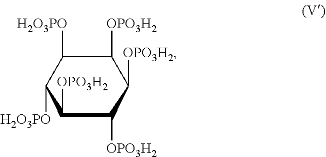

(which corresponds to an additive of formula (V) where M is H and m is 1), this additive advantageously being commercially available e.g. from Nutriscience®,
glucose phosphate, of following formula (VI′):

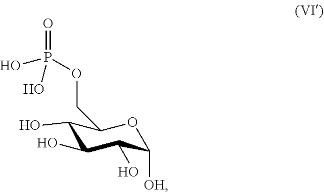

(which corresponds to an additive of formula (VI) where M is H and m is 1), this additive advantageously being commercially available.

Advantageously, phytic acid is an additive improving the dispersibility of the pigment within an aqueous composition.

A method for preparing a pigment pretreated with an additive such as defined above is described for example in WO2012/120098.

In this first embodiment according to the second alternative using a pretreated pigment, the pretreated pigment may or may not then undergo a grinding step. This grinding can limit, even remove aggregates of pretreated pigments thereby facilitating subsequent incorporation thereof in the phase(s) and/or contributing towards reducing sedimentation of the pigment in the phase(s) in which they are contained.

This grinding step can be carried out in the presence of a binder or without a binder (dry grinding).

The binder for example is glycerine, propanediol, a hydrolysate of hydrogenated starch, octyldodecanol, castor oil, a mineral oil, isononyl isononanoate, dimethicone and cyclomethicone, isododecane, and mixtures thereof.

Preferably, when the pigment is treated with an additive improving the dispersibility thereof within an oily phase, the binder is chosen from among glycerine, octyldodecanol, castor oil, a mineral oil, isononyl isononanoate, dimethicone and cyclomethicone, isododecane, and mixtures thereof.

Preferably, when the pigment is not pretreated or when it is treated with an additive improving the dispersibility thereof within an aqueous phase, the binder is chosen from among propanediol, glycerine, a hydrolysate of hydrogenated starch, and mixtures thereof.

The grinding mill is then typically chosen from among three-roll mills, ball mills and disc mills.

If the grinding step is carried out without a binder, the grinding mill can be a pin mill, jet mill micronizer, impact crusher, hammer mill, cutting mill, ball mill, vibrating crusher or cryomill.

In a second embodiment of the second alternative, the at least one pigment is not pretreated with an additive improving the dispersibility thereof, and the method then comprises a grinding step of the pigment. This grinding allows the limiting, even removal of aggregates of pigment, thereby facilitating subsequent incorporation thereof in the phase(s) and/or contributing towards reducing sedimentation of the pigment in the phase(s) in which they are contained.

The embodiments described above for grinding are evidently applicable to this grinding (type of mill, with or without a binder).

Advantageously, a dispersion of the invention comprises between 1% and 60%, preferably between 5% and 50%, in particular between 10% and 40%, better still between 15% and 35%, and preferably between 20% and 25% by weight of pigment(s) relative to the total weight of the dispersed fatty phase in which they are contained.

A dispersion of the invention is advantageous in that the aforementioned advantageous properties thereof remain even in the presence of high contents of pigment in the dispersed fatty phase. Therefore, a dispersion of the invention advantageously has a content higher than or equal to 25%, preferably between 24% and 60%, in particular between 25% and 60%, preferably between 30% and 55%, in particular between 35% and 50%, and better still between 40% and 45% by weight of pigment(s) relative to the total weight of the dispersed fatty phase in which they are contained.

Advantageously, when a phase other than the fatty phase (i.e. droplets (G1), also comprises at least one pigment, in particular the continuous aqueous phase, a dispersion of the invention comprises between 0% and 60%, preferably between 5% and 55%, in particular between 10% and 50%, and better still between 15 and 40% by weight of pigment(s) relative to the total weight of said phase.

Additionally against all expectations, the inventors have observed a particularly advantageous operating range based on the weight ratio of «oil(s)/pigment(s)» which is preferably between 1.2 and 2.1, preferably between 1.3 and 2, in particular between 1.4 and 1.9, and better still between 1.5 and 1.8, preferably between 1.6 and 1.7.

Regarding oils, this weight ratio solely considers «free» or «available» oils. By «free oil» or «available oil», it is meant to designate an oil not previously associated with another raw material, for example similar to EMC30 (INCI: Castor Oil/IPDI Copolymer (and) Caprylic/Capric Triglyceride), which is a pre-mixture of Estogel M in Caprylic/Capric Triglyceride oil in a ratio of 30:70.

A fatty phase having a weight ratio of «oil(s)/pigment(s)» of less than 1.2 has high viscosity, which leads to the formation of deformed droplets, even the impossible implementing of the microfluidic system, in particular if it is sought to produce a dispersion high in dispersed fatty phase. Without wishing to be bound by any theory, the inventors are of the opinion that a fatty phase having a weight ratio «oil(s)/pigment(s)» of less than 1.2. does not provide sufficient availability of oil(s) to guarantee good drop formation.

Also, a fatty phase having a weight ratio of «oil(s)/pigment(s)» higher than 2.1 has low viscosity which leads to slow setting of the fatty phase and hence to the onset of phenomena of droplet fragmentation. In addition, said fatty phase can lead to dispersions of the invention which, when applied to the skin, have a long drying time and therefore possiblly insufficient capability of adhering to the skin.

### Lipophilic Gelling Agents

Advantageously, the fatty phase of a dispersion of the invention also comprises at least one lipophilic gelling agent. Said lipophilic gelling agent differs from the anionic and cationic polymers, oils and pigments described above. This lipophilic gelling agent particularly allows adaptation of viscosity and/or reducing even preventing sedimentation of said pigment(s) at ambient temperature and atmospheric pressure.

This lipophilic gelling agent also allows an increase in the mechanical strength of the droplets as described in WO2017046305.

In the invention, by «gelling agent», it is meant an agent which, at ambient temperature and atmospheric pressure, allows the viscosity of the phase(s) in which it is contained to be increased compared with the same phase(s) not containing said gelling agent, and can for example reach a final viscosity of said phase(s) higher than 2 000 mPa·s, preferably higher than 4 000 mPa·s, better still higher than 10 000 mPa·s, and more particularly higher than 100 000 mPa·s.

Preferably, the viscosity of a phase in the presence of said gelling agent is between 2 000 and 100 000 000 mPa·s, preferably between 4 000 and 1 000 000 mPa·s, and better still from 10 000 to 500 000 mPa·s, at 25° C.

By «lipophilic gelling agent», it is meant a liposoluble or lipodispersible compound able to gel the fatty (or oily) phase of a dispersion of the invention.

In one particular embodiment, the gelling agent is heat-sensitive. The expression «heat-sensitive gelling agent» designates an agent able to increase the viscosity of the fatty phase in which it is contained compared with a fatty phase not including this agent, this viscosity reversibly changing as a function of temperature.

In one embodiment, the gelling agent is chosen from among organic or mineral, polymeric or molecular lipophilic gelling agents; fats solid at ambient temperature and pressure, chosen in particular from among waxes, pasty fats, butters; and mixtures thereof, and is preferably chosen from the group formed by polymeric lipophilic gelling agents.

### Lipophilic Gelling Agent(s)

A lipophilic gelling agent can be chosen from among organic or mineral, polymeric or molecular agents; fats solid at ambient temperature and pressure, chosen in particular from among waxes, pasty fats, butters; and mixtures thereof. Said lipophilic gelling agents are particularly described in WO2019002308.

As mineral lipophilic gelling agent, mention can be made of optionally modified clays such as hectorites modified by a C₁₀ to C₂₂ ammonium chloride such as hectorite modified by distearyl dimethyl ammonium chloride e.g. the one marketed under the trade name Bentone 38VR by ELEMENTIS. Mention can also be made of hectorite modified by distearyldimethylammonium chloride also known as quaternium-18 bentonite, e.g. the product marketed or produced under the trade names Bentone 34 by Rheox, Claytone XL, Claytone 34 and Claytone 40 marketed or produced by Southern Clay, modified clays known under the name benzalkonium bentonite and quaternium-18 bentonite marketed or produced under the trade names Claytone HT, Claytone GR and Claytone PS by Southern Clay, clays modified by stearyldimethylbenzoylammonium chloride, known as stearalkonium bentonites e.g. the products marketed or produced under the trade names Claytone APA and Claytone AF by Southern Clay, and Baragel 24 marketed or produced by Rheox.

Fumed silica can also be cited, optionally with hydrophobic surface treatment having a particle size of less than 1 μm. It is possible chemically to modify the surface of silica by chemical reaction generating a reduction in the number of silanol groups on the silica surface. Silanol groups can especially be substituted by hydrophobic groups: to give a hydrophobic silica.

The hydrophobic groups can be:
trimethylsiloxyl groups, that are particularly obtained by treating fumed silica in the presence of hexamethyldisilazane. Silicas thus treated are called «Silica silylate» according to CTFA (8th Edition, 2000). They are marketed for example under the references Aerosil R812® by DEGUSSA, and CAB-O-SIL TS-530® by CABOT; or
dimethylsilyloxyl or polydimethylsiloxane groups, obtained in particular by treating fumed silica in the presence of polydimethylsiloxane or dimethyldichlorosilane. Silicas thus treated are called «Silica dimethyl silylate» according to CTFA (8th Edition, 2000). They are marketed for example under the references Aerosil R972®, Aerosil R974® by DEGUSSA, CAB-O-SIL TS-610®, and CAB-O-SIL TS-720® by CABOT.

Hydrophobic fumed silica particularly has a particle size than can be nanometric to micrometric, for example ranging from about 5 to 200 nm.

Polymeric organic lipophilic gelling agents are elastomer organopolysiloxanes for example, partially or fully crosslinked, of three-dimensional structure such as those marketed under the trade names KSG6®, KSG16® and KSG18® by SHIN-ETSU, Trefil E-505C″ and Trefil E-506CR by DOW-CORNING, Gransil SR-CYC®, SR DMF10®, SR-DC556®, SR 5CYC gel®, SR DMF 10 gel® and SR DC 556 gel® by GRANT INDUSTRIES, SF 1204® and JK 113® by GENERAL ELECTRIC; ethylcellulose such as sold under the trade name Ethocel® by DOW CHEMICAL; galactomannans comprising one to six and in particular two to four hydroxyl groups per monosaccharide, substituted by a saturated or unsaturated alkyl chain such as guar gum alkylated by C₁ to C₆ alkyl chains and in particular C₁ to C₃ and mixtures thereof. Block copolymers of «diblock», «triblock» or «star» type, of polystyrene/polyisoprene, polystyrene/polybutadiene types such as those marketed under the trade name Luvitol HSB® by BASF, of polystyrene/copoly(ethylene-propylene) type such as those marketed under the trade name Kraton® by SHELL CHEMICAL CO or of polystyrene/copoly(ethylene-butylene) type, mixtures of triblock and star copolymers in isododecane such as those marketed by PENRECO under the trade name Versagel® e.g. the mixture of triblock copolymer butylene/ethylene/styrene and star copolymer ethylene/propylene/styrene in isododecane (Versagel M 5960). Among the lipophilic gelling agents able to be used in the present invention, mention can also be made of esters of dextrin and fatty acids such as dextrin palmitates. Among the esters of dextrin and fatty acid(s) mention can be made for example of dextrin palmitates, dextrin myristates, dextrin palmitates/ethylhexanoates, and mixtures thereof. The esters of dextrin and fatty acid(s) can particularly be cited marketed under the trade names Rheopearl® KL2 (INCI name: dextrin palmitate), Rheopearl® TT2 (INCI name: dextrin palmitate ethylhexanoate), and Rheopearl® MKL2 (INCI name: dextrin myristate) by Miyoshi Europe, also the dextrin palmitate marketed by The Innovation Company. Among polymeric gelling agents, mention can be made of THIXCIN® R by Elementis Specialties (INCI: Trihydroxystearin), OILKEMIA™ 5S polymer by Lubrizol (INCI: Caprylic/Capric Triglyceride (and) Polyurethane-79) or Estogel M by PolymerExpert (INCI: CASTOR OIL/IPDI COPOLYMER & CAPRYLIC/CAPRIC TRIGLYCERIDE). Among waxes and butters, particular mention can be made of C₁₀-C₁₈ triglycerides (INCI: C₁₀-18 Triglycerides) comprising a liquid fraction and a solid fraction at a temperature of 25° C. and at atmospheric pressure (760 mm Hg), shea butter, Nilotica shea butter (Butyrospermum parkii), Galam butter (Butyrospermum parkii), Borneo butter or fat-tengkawang tallow) (Shorea stenoptera), Shorea butter, Illlipé butter, Madhuca or Bassia Madhuca longifolia butter, mowrah butter (Madhuca Latifolia), Katiau butter (Madhuca mottleyana), Phulwara butter (M. butyracea), mango butter (Mangifera indica), Murumuru butter (Astrocatyum murumuru), Kokum butter (Garcinia Indica), Ucuuba butter (Virola sebifera), Tucuma butter, Painya butter (Kpangnan) (Pentadesma butyracea), coffee butter (Coffea arabica), apricot butter (Prunus Armeniaca), Macadamia butter (Macadamia Temifolia), grapeseed butter (Vitis vinifera), avocado butter (Persea gratissima), olive butter (Olea europaea), sweet almond butter (Prunus amygdalus dulcis), cocoa butter (Theobroma cacao) sunflower seed butter, the butter having the INCI name Astrocaryum Murumuru Seed Butter, the butter having the INCI name Theobroma Grandiflorum Seed Butter, and the butter having the INCI name Irvingia Gabonensis Kernel Butter, the esters of jojoba (mixture of jojoba wax and hydrogenated oil) INCI name: Jojoba esters) and the ethyl esters of shea butter (INCI name: Shea butter ethyl esters), and mixtures thereof. In one particular embodiment, the oily phase does not comprise an elastomer gel comprising at least one dimethicone, in particular such as marketed by NuSil Technology under the trade name CareSil™ CXG-1104 (INCI: Dimethicone (and)Dimethicone/Vinyl Dimethicone Crosspolymer).

Preferably, the lipophilic gelling agent is chosen from among dextrin palmitates, Estogel M by PolymerExpert (INCI: CASTOR OIL/IPDI COPOLYMER & CAPRYLIC/CAPRIC TRIGLYCERIDE), OILKEMIA™ 5S polymer by Lubrizol (INCI: Caprylic/Capric Triglyceride (and) Polyurethane-79), and mixture thereof.

Advantageously, a lipophilic gelling agent is a heat-sensitive gelling agent, namely which reacts to heat, and in particular a gelling agent solid at ambient temperature and liquid at a temperature higher than 40° C., preferably higher than 50° C. Advantageously, a lipophilic gelling agent is a thixotropic gelling agent or able to impart thixotropic behaviour to the phase in which it is contained. Said thixotropic gelling agent is chosen in particular from among fumed silicas optionally with hydrophobic treatment as previously described.

In the invention, a dispersion of the invention may comprise from 0.5% to 70%, preferably 1% to 60%, in particular 1.5% to 50%, better still 2% to 40%, in particular 5% to 30%, and more preferably 10% to 20% by weight of lipophilic gelling agent(s) relative to the total weight of the fatty phase(s) in which they are contained.

Evidently those skilled in the art will take care to adjust the parameters of the production method to guarantee good conducting thereof, in particular to ensure the obtaining of phases having adapted fluidity which can be reached in particular through raising the temperature of said phases. These adjustments lie within the general knowledge of skilled persons.

### Additional Compound(s)

In the invention, the continuous aqueous phase and/or fatty phase of a dispersion of the invention nay also comprise at least one additional compound differing from the aforementioned anionic and cationic polymers, oils, pigments, and lipophilic gelling agents.

### Blur Effect Filler/«Soft-Focus» Fillers

The continuous aqueous phase and/or dispersed fatty phase, in particular the dispersed fatty phase, of a dispersion of the invention may further comprise at least one blur effect filler.

A blur effect filler is able to modify and/or hide wrinkles via the intrinsic physical properties thereof. These fillers in particular can modify wrinkles via a tensor effect, camouflage effect, or blur effect.

As blur effect filler, the following examples of compounds can be given:
silica porous microparticles e.g. Silica Beads® SB 150 and SB 700 by Miyoshi having a mean size of 5 μm, and SUNSPHERES® H series by Asahi Glass such as H33, H51, of size 3.5 and 5 μm respectively, and Sensibead Si 175 and Sensibead Si 320 by Sensient Cosmetic Technologies of size 7 μm and 5 μm respectively;
hollow hemispherical particles of silicone resins such as NLK 500®, NLK 506® and NLK 510@ by Takemoto Oil and Fat, described in particular in EP 1 579 849;
silicone resin powders such as SILICON Resin Tospearl® 145 A DE GE silicone of mean size 4.5 μm;
powders of acrylic copolymers in particular of methyl poly(meth) acrylate, e.g. the particles PMMA Jurimer MBI® by Nihon Junyoki of mean size 8 μm, hollow PMME spheres sold under the trade name COVABEAD® LH 85 by Sensient Cosmetic Technologies, and expanded microspheres of vinylidene/acrylonitrile/methylene methacrylates sold under the trade name Expancel®;
wax powders such as Paraffin wax microloase® 114S particles by Micropowders of mean size 7 μm;
polyethylene powders particularly comprising at least one ethylene/acrylic acid copolymer such as FLOBEADS® EA 209 E by Sumimoto of mean size 10 μm;
powders of elastomer organopolysiloxanes, crosslinked and coated with silicone resin, in particular silsesquioxane under the trade names KSP 100®, KSP 101@, KSP 102®, KSP 103@, KSP 104@ and KSP 105@ by Shin Etsu;
composite powders of talc/titanium dioxide/alumina/silica such as Coverleaf AR 80@ by Catalyst & Chemical;
talc, mica, kaolin, lauryl glycine, starch powders crosslinked by octenyl succinate anhydride, boron nitride, polytetrafluoroethylene powders, precipitated calcium carbonate, the carbonate of magnesium hydrogencarbonate, barium sulfate, hydroxyapatite, calcium silicate, cerium dioxide and glass or ceramic microcapsules;
hydrophilic or hydrophobic fibres, synthetic or natural, mineral or organic, such as fibres of silk, cotton, wool, flax, cellulose extracted in particular from wood, vegetables or algae, polyamide (Nylon®), modified cellulose, poly-p-phenylene terephthalamide, acrylic, polyolefin, glass, silica, aramid, carbon, polytetrafluoroethylene (Téflon®), insoluble collagen, polyesters, vinyl or vinylidene polychloride, polyvinyl alcohol, polyacrylonitrile, chitosan, polyurethane, polyethylene phthalate, fibres formed from a mixture of polymers, resorbable synthetic fibres, and mixtures thereof described in patent application EP 1 151 742;
spherical elastomer crosslinked silicones such as Trefil E-505C® or E-506 C® by Dow Corning;
abrasive fillers which, via mechanical effect, provide smoothing of skin microrelief such as abrasive silica e.g. Abrasif SP® by Semanez, or powders of walnut or nut shells (apricot, walnut e.g. by Cosmétochem); and
mixtures thereof.

Fillers having an effect signs of ageing are chosen in particular from among porous silica microparticles, hollow hemispherical particles of silicones, silicone resin powders, acrylic copolymer powders, polyethylene powders, elastomer organopolysiloxanes powders crosslinked and coated with silicone resin, composite powders of talc/titanium dioxide/alumina/silica, precipitated calcium carbonate, the carbonate of magnesium hydrogencarbonate, barium sulfate, hydroxyapatite, calcium silicate, cerium dioxide and glass or ceramic microcapsules, silk fibres, cotton fibres, and mixtures thereof.

### Colouring Agent

The continuous phase and/or dispersed phase, in particular the fatty phase, can also comprise at least one colouring agent differing from the above-mentioned pigment(s) and fillers.

A colouring agent can be chosen in particular from among colourants that are water-soluble or not, liposoluble or not, organic or inorganic, optical effect materials, liquid crystals and mixtures thereof.

In particular, a colouring agent can be a colourant and/or pearl e.g Covapearl Star sparkling gold 2375 by Sensient Cosmetic Technologies or Covapearl antique silver 239 by Sensient Cosmetic Technologies. Preferably a colourant or pearl is chosen having a different colour from the pigment used. By «colourant», it is meant a colouring chemical substance soluble in the coloured particle (or phase of the coloured particle in which the colourant is contained). By «soluble», it is meant that the solubility at 20° C. of the colourant in the coloured particle is higher than 2 g/L, in particular higher than 5 g/L, preferably higher than 10 g/L.

Preferably, when the dispersion of the invention is multiphase, the phase comprising the pigment(s) differs from the phase comprising the pearl(s) and/or colourants. This gives rise to an enhanced and even unexpected visual effect for the consumer who, in one particular embodiment, sees that the colour of the product in the container (the colour of the pigment(s) of the dispersed fatty phase) differs from the expected colour (the colour of the pearl(s) and/or colourant(s)).

Additionally, the continuous phase and/or dispersed phase, in particular the fatty phase, of a dispersion of the invention may also comprise powders: glitter; reflective particles (i.e. particles of which the size, structure, in particular thickness of the constituent layer(s) and the physical and chemical nature and surface condition allow the reflecting of incident light). This reflection can if desired, have sufficient intensity to create highlight areas on the surface of the dispersion or composition of the invention when applied to a make-up surface, this highlighting being visible to the naked eye i.e. brighter areas contrasting with their environment and appearing to shine); particulate agents insoluble in the fatty phase; emulsifying or non-emulsifying silicone elastomers, in particular those described in EP2353577; preserving agents; humectants; stabilizers; chelating agents; film-forming polymers (i.e. polymers able alone, or in the presence of an auxiliary filming agent, to form a continuous film adhering to a substrate in particular to keratin materials and in particular the skin); auxiliary filming agents such as aforementioned; emollients; modifying agents chosen from among texturizing agents, viscosifying agents (e.g. aqueous phase gelling/texturizing agents differing from the above-mentioned base), pH modifiers, modifiers of osmotic force and/or modifiers of refractive index etc . . . or any usual cosmetic additive; and mixtures thereof.

In one embodiment, the particulate agents insoluble in the droplet fatty phase are chosen from the group formed by ceramics, polymers, in particular acrylic polymers, and mixtures thereof.

### Hydrophilic Texturizing Agent

In the remainder of the present description, a hydrophilic texturizing agent can indifferently be termed a «hydrophilic gelling agent».

Depending on the fluidity of the dispersion it is desired to obtain, one or more hydrophilic texturizing agents can be incorporated in the dispersion of the invention, in particular in the continuous aqueous phase.

As hydrophilic texturizing agents i.e. soluble or dispersible in water, and hence able to be included in the aqueous phase of a dispersion of the invention, mention can be made of:
natural texturizing agents, chosen in particular from among algae extracts, plant exudates, seed extracts, microorganism fermentation products such as alcasealan (INCI: Alcaligenes Polysaccharides), and other natural agents in particular hyaluronic acid,
semi-synthetic texturizing agents chosen in particular from among cellulose derivatives and modified starches,
synthetic texturizing agents chosen in particular from among homopolymers of (meth)acrylic acid or ester thereof, copolymers of (meth)acrylic acid or ester thereof, AMPS copolymers (2-acrylamido-2-methylpropane sulfonic acid), associative polymers,
other texturizing agents chosen in particular from among polyethyleneglycols (marketed under the trade name Carbowax), clay, silicas such as those marketed under the trade names Aerosil® 90/130/150/200/300/380), glycerine, and
mixtures thereof.

By «associative polymer» in the meaning of the invention, it is meant any amphiphilic polymer having in its structure at least one fatty chain and at least one hydrophilic portion; the associative polymers conforming to the present invention can be anionic, cationic, non-ionic, or amphoteric; in particular they are those described in FR 2 999 921.

These hydrophilic texturizing agents are described in more detail in FR3041251.

In addition, these hydrophilic texturizing agents can reinforce the kinetic stability of a dispersion of the invention, in particular when the continuous aqueous phase is liquid at ambient temperature and atmospheric pressure.

The continuous phase and/or dispersed phase, in particular the fatty phase, of a dispersion of the invention can further comprise at least one active substance, particularly biological or cosmetic active substances, preferably chosen from among hydrating agents, healing agents, depigmenting agents, UV filters, peeling agents, antioxidants, active substances stimulating the synthesis of dermal and/or epidermal macromolecules, dermo-relaxant agents, antiperspirant agents, soothing agents, anti-ageing agents, fragrances and mixtures thereof.

Preferably, a dispersion of the invention further comprises UV filters such as those described in particular in FR3041251.

Evidently, those skilled in the art will take care to choose any optional additional compound(s) and/or active substances mentioned above and/or the respective amounts thereof so that the advantageous properties of a dispersion of the invention are not or not substantially deteriorated by the envisaged addition. In particular, the type and/or amount of the additional compound(s) and/or active substance(s) are dependent on the aqueous or oily nature of the phase under consideration and/or on the method used (in particular of «non-microfluidic type» or «microfluidic type»). These adjustments are within the reach of persons skilled in the art.

### Preparation Method

The dispersions of the invention can be prepared with different methods.

Therefore, the dispersions of the invention have the advantage that they can be prepared with a simple «non-microfluidic» method, namely simply by emulsification.

As for a conventional emulsion, an aqueous solution and a fatty solution are prepared separately. It is the addition under agitation of the fatty phase to the aqueous phase which creates the direct emulsion and hence the dispersion of the invention.

The viscosity of the aqueous phase can be controlled, in particular by acting on the quantity of anionic polymer (carbomer in particular) and the pH of the solution. In general, the pH of the aqueous phase is lower than 4.5 which may necessitate the addition of a third solution, sodium hydroxide (BF), at a last stage to reach a pH of between 5.5 and 6.5.

The viscosity of the aqueous phase and the shear force applied to the mixture are the two main parameters which impact the size (and hence macroscopic nature) and monodispersity of the droplets of the dispersion of the invention.

Persons skilled in the art are able to adjust the non-microfluidic method to meet the criterion of mean diameter of the droplets of the dispersion of the invention.

The different fluids, in particular the flow rates of the fluids, can be used in a microfluidic method of the invention by applying a hydrodynamic mode known as «dripping» or «jetting» (forming of a liquid jet at the exit of the microfluidic device followed by fragmentation of the jet in ambient air under the effect of gravity).

The dispersions of the invention can also be prepared using a microfluidic method. Microfluidic methods able to produce dispersions of the invention are particularly described in WO2012/120043, WO2015/055748 or WO2019145424.

In one preferred embodiment, the production method is based on a microfluidic method such as described in WO2019145424, namely in which the forming of the droplets is obtained by means of a nozzle able to convey a fluid jet formed of a second fluid concentrically surrounding a first fluid, and a mechanical fragmentation device of said fluid jet arranged in the vicinity of the exit from the nozzle. In this embodiment, the droplets obtained with a microfluidic method display a uniform size distribution with high production yield.

Preferably, the dispersions of the invention are composed of a population of monodisperse droplets G1 in particular such that they have a mean diameter D̅ of between 150 μm and 3 000 μm and a coefficient of variation Cv of less than 10%, even less than 3%.

In the present description, by “monodisperse droplets” it is meant the fact that the population of droplets G1 of the dispersion of the invention has a uniform size distribution. Conversely, droplets having poor monodispersity are said to be “polydisperse”.

In one mode, the mean diameter D̅ of the droplets is measured for example by photographic analysis of a batch composed of N droplets using image processing software (Image J). Typically, with this method, the diameter is measured in pixels then related to μm, as a function of the size of the receptacle containing the droplets of the dispersion.

Preferably, the value of N is chosen to be higher than or equal to 30, so that this analysis reflects the distribution of the diameters of the droplets of said emulsion in statistically significant manner. N is advantageously higher than or equal to 100, in particular for a dispersion that is polydisperse.

The diameter Di of each droplet is measured, to obtain the mean diameter D by calculating the arithmetic mean of these values:

$$\overline{D} = \frac{1}{N} {\sum }_{i = 1}^{N} {D}_{i}$$

From these values Dᵢ, it is also possible to obtain the standard deviation σ of the diameters of the droplets in the dispersion:

$$σ = \sqrt{\frac{{\sum }_{i = 1}^{N} {( {D}_{i} - \overline{D} )}^{2}}{N}}$$

The standard deviation σ of a dispersion reflects the distribution of the diameters Dᵢ of the droplets in the dispersion around the mean diameter D̅.

With knowledge of the mean diameter D̅ and standard deviation σ of a dispersion, it is possible to determine that 95.4% of the population of droplets lies within the range of diameters [D̅−2σ: D̅+2σ] and that 68.2% of the population lies within the range [D̅−θ;D̅+σ]

To characterize the monodispersity of the dispersion in this embodiment of the invention, the coefficient of variation can be calculated:

$${C}_{v} = \frac{σ}{\overline{D}}$$

This parameter reflects the distribution of the diameters of the droplets as a function of the mean diameter thereof.

The coefficient of variation Cv of the diameters of the droplets G1 according to this method of the invention is lower than 10%, preferably lower than 5%, even lower than 3%.

Alternatively, monodispersity can be evidenced by placing a sample of the dispersion in a bottle having a constant circular cross-section. Gentle agitation by rotating the bottle over a quarter of a turn in one half-second about the axis of symmetry passing through the bottle, followed by a rest time of one half-second is performed before repeating the operation in the opposite direction, the whole operation being repeated four successive times.

The droplets of the dispersed phase organize themselves into crystalline form if they are monodisperse. They therefore exhibit a stack pattern that is repeated in the three dimensions. It is then possible to observe regular stacking which indicates good monodispersity, irregular stacking translating polydispersity of the dispersion.

To obtain monodisperse droplets, use can also be made of the microfluidic technique in (Utada et al. MRS Bulletin 32, 702-708 (2007); Cramer et al. Chem. Eng. Sci. 59, 15, 3045-3058 (2004)), and more particularly using microfluidic devices of co-flow type (the fluids flow in the same direction) or flow-focusing type (the fluids flow in different directions and typically in opposite directions).

The presence in the fatty phase of gelling agent(s) such as previously described, in particular heat-sensitive, may require adjustments to the method for preparing a dispersion of the invention. In particular, the method for preparing a dispersion of the invention may comprise a heating step (between 40° C. and 150° C., in particular between 50° C. and 90° C.) at least of the fatty phase and optionally of the aqueous phase, before mixing/placing in contact said fatty phase with the aqueous phase, and optionally—if a «non-microfluidic» method is used such as mentioned above-maintaining this heating during agitation until the desired dispersion is obtained.

In one embodiment, the preparation method of the dispersions of the invention comprises a step to form droplets, comprising:
optionally, heating an oily fluid FI and/or aqueous fluid FE, to a temperature of between 40° C. and 150° C.;
contacting an aqueous fluid FE with an oily fluid FI such as defined below; and
forming droplets of a fatty phase, composed of the oily fluid FI, dispersed in a continuous aqueous phase composed of fluid FE, said droplets comprising a shell isolating the core of the fatty phase droplets of the dispersion.

In one embodiment, the fluid FI is initially prepared by mixing at least one oil and at least one pigment and at least one cationic polymer such as previously defined, in particular amodimethicone, and optionally at least one lipophilic gelling agent and/or at least one additional compound such as mentioned above, on the understanding that the quantity of amine functions contributed by the cationic polymer in the fatty phase is between 10.8 μmol and 32.4 μmol per gram of fatty phase.

In one embodiment, the fluid FE comprises at least water and at least one anionic polymer such as previously defined, in particular a carbomer, and optionally at least one hydrophilic texturizing agent, a base, at least one additional compound, preserving agents and/or other water-soluble products such as glycerine such as aforementioned.

In one embodiment, the continuous aqueous phase of the formed dispersion comprises, and is even composed of, the aqueous phase of fluid FE. The anionic polymer contained in said fluid FE serves chiefly to form the shell of the droplets. Said anionic polymer also contributes to increasing the viscosity of the fluid FE, and hence that of the continuous aqueous phase.

In one embodiment, a method of the invention and in particular the droplet formation step, may also comprise a step to inject a solution increasing the viscosity of the continuous aqueous phase of the fluid FE. Preferably, the viscosity-increasing solution is aqueous. This viscosity-increasing solution is typically injected into the external aqueous fluid FE after formation of the dispersion of the invention, and therefore after formation of the droplets.

In one embodiment, the viscosity-increasing solution comprises a base, in particular an alkaline hydroxide such as sodium hydroxide.

In one embodiment, the temperature of the above-mentioned heating step is from 50° C. to 80° C., preferably 50° C. to 70° C., and more preferably 55 to 65° C.

Depending on the pigment(s) used, a method for preparing a dispersion of the invention may comprise the steps of:
a) providing at least one pigment, optionally pretreated with an additive improving the dispersibility of the pigment, then
b) optionally grinding said at least one pigment, said grinding preferably taking place when the at least one pigment is not pretreated,
c) dispersing the at least one pigment in at least one oily fluid FI,
e) optionally, heating said oily fluid FI and optionally the aqueous fluid FE, to a temperature of 40° C. to 150° C., preferably 50° C. to 90° C.;
f) placing the aqueous fluid FE and oily fluid FI in contact; and
g) forming droplets of a fatty phase composed of the oily fluid FI, dispersed in a continuous aqueous phase composed of the aqueous fluid FE, said droplets optionally comprising a shell isolating the core of the fatty phase droplets of the dispersion,
wherein, the oily fluid FI and aqueous fluid FE are such as previously described.

### Uses

Preferably, a dispersion of the invention can be used directly after the aforementioned preparation methods, as a composition and in particular a cosmetic composition. The dispersion of the invention, when prepared with a microfluidic method such as described above, can also be used as composition, in particular cosmetic composition, after separation of the droplets and redispersion thereof in a suitable second phase.

The invention also concerns the use of at least one dispersion of the invention for inclusion in a cosmetic composition.

The dispersions of the invention can be used in particular in the cosmetic field.

The invention also concerns a cosmetic composition, preferably a make-up composition, comprising at least one dispersion such as defined above.

The cosmetic compositions of the invention, in addition to the above-mentioned ingredients, may also comprise at least one physiologically acceptable medium.

The invention therefore also concerns a composition comprising at least one dispersion such as defined above in association with a physiologically acceptable medium.

By “physiologically acceptable medium”, it is meant to designate a medium particularly suitable for the application of a composition of the invention to keratin material, in particular the skin, lips, nails, eyelashes or eyebrows, and preferably the skin.

The physiologically acceptable medium is generally adapted to the type of substrate on which the composition is to be applied, and to how the composition is to be packaged.

The presence of a physiologically acceptable medium can contribute towards improving the preserving and/or maintained integrity over time of the droplets of a dispersion of the invention.

In one embodiment, the physiologically acceptable medium is in the form of an aqueous gel having adapted viscosity, in particular to ensure suspending of the droplets of the invention.

In one embodiment, the cosmetic compositions are used for the make-up and/or care of keratin material, the skin in particular.

The cosmetic compositions of the invention can be care products, sunscreen products, cleansing products (make-up removal), hygiene or make-up products.

These compositions are therefore intended to be applied in particular to the skin, lips or hair.

Therefore, the present invention also concerns the non-therapeutic cosmetic use of a dispersion or composition of the invention, as make-up, hygiene, cleansing and/or care product of keratin material, in particular the skin.

In one embodiment, the dispersions or compositions of the invention are in the form of a foundation, make-up remover, face care and/or body care and/or hair care product, an anti-ageing, sunscreen, oily skin, whitening or hydrating care product, a BB cream, tinted cream or foundation, face and/or body cleanser, shower gel or shampoo, preferably a foundation.

A dispersion or composition of the invention can in particular be a sun composition, care cream, serum or deodorant.

The dispersions or compositions of the invention can be in various forms, in particular in the form of a cream, balm, lotion, serum, gel, gel-cream or a mist.

In particular, a dispersion or composition of the invention is a care and/or make-up composition for keratin material, in particular the skin, and particularly a make-up composition.

More particularly, a dispersion or composition of the invention can be a mascara for example, a skin complexion product such as a foundation, an eyeliner, eyeshadow or blush, a product for the lips such as lipstick or lip-gloss, an optionally liquid soap, shampoo, conditioner, nail varnish, preferably an eyeshadow, skin complexion product or products for the lips. The dispersion or composition of the invention can be in the form of a monophasic or biphasic lotion, an emulsion, gel, stick or cream.

A dispersion or composition of the invention is preferably in the form of a foundation to be applied to the face or neck, a concealer product, colour corrector, a tinted cream, or make-up base for the face or make-up composition for the body.

The present invention also concerns a non-therapeutic cosmetic treatment method, in particular make-up and/or care method, preferably make-up method of a keratin material in particular the skin, lips or hair, comprising at least one step to apply to said keratin material at least one dispersion or composition of the invention.

In particular, the present invention concerns a non-therapeutic cosmetic treatment method, in particular make-up method, of the skin comprising a step to apply to the skin at least one dispersion or composition of the invention.

In the entire description, including in the claims, the expression «comprising one» is to be understood as being synonymous with «comprising at least one», unless otherwise specified.

The expressions «between . . . and . . .», «from . . . to . . .» and «ranging from . . . to . . .» are to be understood as including the limits, unless otherwise specified.

The quantities of ingredients given in the examples are expressed as percentage by weight relative to the total weight of the composition, unless otherwise specified.

The following examples illustrate the present invention but do not limit the scope thereof.

### EXAMPLES

Unless otherwise indicated, the compositions described below were obtained following a microfluidic method such as described in WO2019145424.

### Example 1: Comparative Pigmented Macroscopic Dispersions

The compositions of the (fluid) phases were as a follows:

| Name | | INCI name | % w/w Phases | |
|---|---|---|---|---|
| AQUEOUS GEL PHASE (=OF) | | | 100.00 | A |
| Osmosis water | / | Aqua | q.s.* | A1 |
| MICROCARE PE | Thor | Phenoxyethanol, aqua | 0.87 | A1 |
| MICROCARE | Thor | Pentylene glycol, aqua | 2.17 | A1 |
| EMOLLIENT PTG | | | | |
| CARBOPOL | Lubrizol | Carbomer | 0.20 | A3 |
| ETD2050 | | | | |
| ALCASEALAN | Hakuto | Alcaligenes | 0.02 | A2 |
| | | polysaccharides | | |
| GLYCERINE | Interchimie | Glycerin, aqua | 3.26 | A4 |
| CODEX | | | | |
| GLUCAM E20 | Lubrizol | Methyl gluceth-20 | 3.26 | A4 |
| HUMECTANT | | | | |
| UNITAMURON | Induchem | Butylene | 5.43 | A5 |
| H-22 | | glycol, tamarindus inca | | |
| | | seed gum, | | |
| | | phenoxyethanol | | |
| EDETA BD | BASF | Disodium EDTA | 0.03 | A1 |
| SODIUM | Panreac | Sodium hydroxide | 0.03 | A6 |
| HYDROXIDE | | | | |
| PELLETS PRS | | | | |
| CODEX | | | | |
| OILY PHASE (=IF) | | | 100.00 | B |
| LABRAFAC CC | Gatefosse | Caprylic/capric | q.s.* | B1/B2 |
| | | triglyceride | | |
| KAK HL | Kokyu alcohol | Hexyl laurate | 28.90 | B2 |
| | kogyo co | | | |
| EMC30 | Polymerexpert | Caprylic/capric | 6.72 | B2 |
| | | triglyceride, Castor | | |
| | | oil/IPDI copolymer, | | |
| | | aqua | | |
| ASL-1 TIO2 CR- | Daito Kasei | CI77891, Aluminum | 30.82 | B3 |
| 50 | | hydroxide, Sodium | | |
| | | Lauroyl glutamate, | | |
| | | Lysine, Magnesium | | |
| | | chloride | | |
| ASL-1 YELLOW | Daito Kasei | CI 77492, Sodium | 4.09 | B3 |
| LL-100P | | Lauroyl glutamate, | | |
| | | Lysine, Magnesium | | |
| | | chloride | | |
| ASL-1 RED R- | Daito Kasei | CI 77491, Sodium | 1.02 | B3 |
| 516P | | Lauroyl glutamate, | | |
| | | Lysine, Magnesium | | |
| | | chloride | | |
| ASL-1 BLACK | Daito Kasei | CI 77499, Sodium | 0.65 | B3 |
| BL-100P | | Lauroyl glutamate, | | |
| | | Lysine, Magnesium | | |
| | | choride | | |
| Fragrance | — | Fragrance | 0.20 | B4 |
| CAS-3131 PILOT | Nusil | Amodimethicone | X | B1 |
| | | | (see Table 1) | |

*q.s.: in sufficient amount.

TABLE 1

| | 1A | 1B | 1C | 1D | 1E | 1F | 1G | 1H | 1I |
|---|---|---|---|---|---|---|---|---|---|
| CAS-3131 | (comp.) | (Inv.) | (Inv.) | (Inv.) | (Inv.) | (Inv.) | (Inv.) | (Inv.) | (comp.) |
| % w/w IF | 0.5 | 0.75 | 1 | 1.25 | 1.5 | 1.75 | 2 | 2.25 | 2.5 |
| Quantity of | 7.2 | 10.8 | 14.4 | 18 | 21.6 | 25.2 | 28.8 | 32.4 | 36 |
| amine functions | | | | | | | | | |
| contributed by | | | | | | | | | |
| the cationic | | | | | | | | | |
| polymer in fatty | | | | | | | | | |
| phase (in μmol/g) | | | | | | | | | |

### Preparation Protocol:

For OF:
A1: Phenoxyethanol, Pentyleneglycol and EDTA are incorporated in the water. The mixture is left under agitation 5 min.
A2: Alcasealan is added under rotor stator stirring (4500 rpm) for 15 min.
A3: The carbomer is dispersed in the preceding mixture under agitation using a dispersion blade.
A4: Glycerine and Glucam E20 are mixed together, and this mixture is added under agitation for 10 min.
A5: Unitamuron H-22 is next added to the mixture under dispersing agitation for 20 min.
A6: Finally, sodium hydroxide is added and the solution is mixed for 10 minutes.

### For IF:

B1: Amodimethicone is added to a portion of KAK HL and mixed with a magnetic stir bar for 5 min (=mixture B1).
B2: Grinding of the pigments is prepared in a portion of Labrafac CC(=mixture B2).
B3: In parallel, EMC30 is mixed with the remainder of Labrafac CC and heated to 85° C. for dispersion thereof (=mixture B3).
B4: Mixture B2 is added to mixture B3 at 80° C. under agitation until homogenization (=mixture B4), and mixture B4 is cooled to 50° C. under agitation.
B5: In parallel, CAS-3131 is mixed with the remainder of KAK HL at 50° C. (=mixture B5).
B6: Mixture B5 is added to mixture B4 under agitation until homogenization, the fragrance is added, and the mixture left to return to AT (=mixture B6).

| Parameters of the microfluidic method | % w/w |
|---|---|
| OF | 72.90% |
| IF | 25.00% |
| BF** | 2.10% |

**Optionally, provision can be made for the addition of a viscosity-increasing solution (BF) to the continuous phase to improve the suspending of the dispersed phase droplets in the continuous phase, in particular as described in WO2015055748. In particular, this solution BF is a sodium hydroxide solution (NaOH).

The entire method and phases used are at ambient temperature.

A macroscopic dispersion is obtained having a high content of droplets of pigmented fatty phase (i.e. 25%) and in which the droplets having a diameter greater than or equal to 150 μm represent a volume greater than or equal to 60%, even greater than or equal to 70% of the total volume of the dispersed phase, and at least 60% of the droplets have a mean diameter greater than or equal to 150 μm, even greater than or equal to 250 μm.

### Results:

Concerning dispersion 1A, it is observed that the fatty phase droplets have low sphericity and mechanical strength. A reduction in the flow rate of the fatty phase in the microfluidic method allowed evidencing of an improvement in sphericity and mechanical strength of the droplets; truly satisfactory results were observed when the content of fatty phase in dispersion 1A is less than or equal to 9% relative to the total weight of the dispersion.

Concerning dispersion 1I, it is observed that the fatty phase has high viscosity detrimental to the proper conducting of the microfluidic method. It is therefore difficult, even impossible, to produce dispersion 1 I using the microfluidic system under consideration.

Concerning dispersions 1B-1H of the invention, these all exhibit unique visual appearance, namely macroscopic pigmented droplets dispersed in a transparent suspensive aqueous phase, the droplets having particularly satisfactory properties in terms of sphericity and mechanical strength. Even better satisfactory results are observed with dispersions 1D-1F, and more particularly with dispersion 1E.

Moreover, on application, the make-up result is progressive (or unfolding); first a pale shade of the skin is observed which gradually intensifies. The final shade appears after about 45 seconds after application to the skin.

In addition to a particularly satisfactory make-up result, the dispersions 1B-1H impart satisfactory sensorial properties when applied to the skin, in particular in terms of freshness and hydration.

### Example 2: Optimized Pigmented Macroscopic Dispersions of the Invention

Unless otherwise stated, the compositions, preparation protocols, method and microfluidic parameters were the same as those described in Example 1.

The compositions of the fatty phases under consideration in Example 2 were the following:

| Name | | INCI name | % w/w Phases |
|---|---|---|---|
| OILY PHASE (=IF) | | | 100.00 |
| LABRAFAC CC | Gatefosse | Caprylic/Capric Triglyceride | X |
| | | | (see Table 2) |
| KAK HL | Kokyu alcohol | Hexyl laurate | 28.90 |
| | kogyo co | | |
| EMC30 | Polymerexpert | Caprylic/Capric Triglyceride, Castor | 6.72 |
| | | oil/IPDI copolymer, aqua | |
| ASL-1 TIO2 CR- | Daito Kasei | CI77891, Aluminum hydroxide, Sodium | 30.82 |
| 50 | | Lauroyl glutamate, Lysine, Magnesium | |
| | | chloride | |
| ASL-1 YELLOW | Daito Kasei | CI 77492, Sodium Lauroyl glutamate, | 4.09 |
| LL-100P | | Lysine, Magnesium chloride | |
| ASL-1 RED R- | Daito Kasei | CI 77491, Sodium Lauroyl glutamate, | 1.02 |
| 516P | | Lysine, Magnesium chloride | |
| ASL-1 BLACK | Daito Kasei | CI 77499, Sodium Lauroyl glutamate, | 0.65 |
| BL-100P | | Lysine, Magnesium choride | |
| Fragrance | — | Fragrance | 0.20 |
| CAS-3131 PILOT | Nusil | Amodimethicone | 1.5 |

* q.s: in sufficient amount

TABLE 2

| Dispersion | 2A | 2B | 2C | 2D | 2E | 2F | 2G | 2H | 2I | 2J | 2K | 2L | 2M |
|---|---|---|---|---|---|---|---|---|---|---|---|---|---|
| Pigments - % w/w | 36.58 | | | | | | | | | | | | |
| IF | | | | | | | | | | | | | |
| Free oils - % w/w | 36.6 | 40.2 | 43.9 | 47.5 | 51.2 | 54.9 | 58.5 | 62.2 | 65.8 | 69.5 | 73.1 | 76.8 | 80.47 |
| IF | | | | | | | | | | | | | |
| Ratio “oils/ | 1.0 | 1.1 | 1.2 | 1.3 | 1.4 | 1.5 | 1.6 | 1.7 | 1.8 | 1.9 | 2.0 | 2.1 | 2.2 |
| pigments” | | | | | | | | | | | | | |

For each dispersion 2A to 2M, the sphericity of the droplets and fragmentation of the droplets were observed.

### Scoring Criteria:

| SCORING | | | | |
|---|---|---|---|---|
| CRITERIA | 0 | 1 | 2 | 3 |
| Droplet | Good | Slightly | Moderately | Highly |
| sphericity | sphericity | elongate | elongate | elongate |
| | | droplets | droplets | droplets |
| Droplet | No | Slight | Moderate | Strong |
| fragmentation | fragmentation | fragmentation | fragmentation | fragmentation |

### Results:

TABLE 2

| Dispersion | 2A | 2B | 2C | 2D | 2E | 2F | 2G | 2H | 2I | 2J | 2K | 2L | 2M |
|---|---|---|---|---|---|---|---|---|---|---|---|---|---|
| Droplet | 3 | 3 | 2 | 2 | 1 | 1 | 0 | 0 | 0 | 1 | 1 | 1 | 1 |
| sphericity | | | | | | | | | | | | | |
| Droplet | 1 | 1 | 1 | 1 | 1 | 0 | 0 | 0 | 1 | 1 | 2 | 2 | 3 |
| fragmen- | | | | | | | | | | | | | |
| tation | | | | | | | | | | | | | |

In the light of the above results, it is observed that:
a fatty phase having a weight ratio «oil(s)/pigment(s)» of less than 1.2 has viscosity that is too high, leading to the formation of highly deformed droplets, even to impossible implementing of the microfluidic system; and
a fatty phase having a weight ratio «oil(s)/pigment(s)» higher than 2.1 is too fluid, leading to the onset of phenomena of droplet fragmentation.

A sensorial test also showed that a fatty phase having an «oil(s)/pigment(s)» weight ratio higher than 2.1 leads to dispersions of the invention which, on application to the skin, have long drying times and therefore a capability of adhering to the skin which may be insufficient.

The dispersions of the invention exhibiting the best results in terms of (i) droplet sphericity (ii) droplet fragmentation (iii) capability of adhering to the skin are those in which the fatty phases have an «oil(s)/pigment(s)» weight ratio of between 1.2 and 2.1 (i.e. dispersions 2C-2L), preferably between 1.4 and 1.9 (i.e. dispersions 2E-2J), and most particularly between 1.6 and 1.7 (i.e. dispersions 2G and 2H).

1. A dispersion comprising a fatty phase in the form of droplets dispersed in a continuous aqueous phase, the dispersed phase and the continuous phase being non-miscible with each other at ambient temperature and atmospheric pressure, wherein the droplets comprise at least one shell and pigments, said shell being formed of at least one anionic polymer comprising at least one carboxylic acid function and of at least one cationic polymer comprising at least two amine functions, wherein the quantity of amine functions contributed by the cationic polymer in the fatty phase is between 10.8 μmol and 32.4 μmol per gram of fatty phase.

2. The dispersion according to claim 1, wherein the quantity of amine functions contributed by the cationic polymer in the fatty phase is between 14.4 μmol and 28.8 μmol per gram of fatty phase.

3. The dispersion according to claim 1, wherein the fatty phase comprises between 1% and 60% by weight of pigment(s) relative to the total weight of the fatty phase.

4. The dispersion according to claim 1, wherein the droplets having a diameter greater than or equal to 150 μm represent a volume greater than or equal to 60% of the total volume of the dispersed phase and/or at least 60% of the droplets have a mean diameter greater than or equal to 150 μm.

5. The dispersion according to claim 1, comprising between 1% and 99.25% by weight of oil(s) relative to the total weight of the fatty phase.

6. The dispersion according to claim 1, wherein the weight ratio «oil(s)/pigment(s)» is between 1.2 and 2.1.

7. The dispersion according to claim 1, wherein the droplets comprise a core that is liquid or at least partly gelled or at least partly thixotropic, said core being monophase or comprising an intermediate droplet of an intermediate phase and at least one inner droplet of an inner phase arranged in the intermediate droplet, the intermediate phase and the inner phase being non-miscible with each other at ambient temperature and atmospheric pressure, the pigment(s) being included in the intermediate phase and/or inner phase.

8. The dispersion according to claim 1, wherein the fatty phase also comprises at least one lipophilic gelling agent.

9. The dispersion according to claim 1, comprising from 0.5% to 70% by weight of lipophilic gelling agent(s) relative to the total weight of the fatty phase in which they are contained.

10. The dispersion according to claim 1, wherein the cationic polymer is a silicone polymer modified by a primary, secondary or tertiary amine function.

11. The dispersion according to claim 1, wherein the anionic polymer is a polymer comprising monomer units comprising at least one chemical carboxylic acid function.

12. The dispersion according to claim 1, wherein the continuous aqueous phase, even said dispersion, does not comprise a surfactant.

13. A method for preparing the dispersion of claim 1, comprising the following steps:
optionally, heating an oily fluid FI and/or an aqueous fluid FE, to a temperature of 40° C. to 150° C.;
contacting the aqueous fluid FE with the oily fluid FI; and
forming droplets of the fatty phase, composed of the oily fluid FI, dispersed in a continuous aqueous phase composed of the aqueous fluid FE, said droplets comprising a shell isolating the core of the fatty phase droplets of the dispersion,
wherein:
the oily fluid FI comprises at least one oil, at least one pigment and at least one cationic polymer, and optionally also at least one lipophilic gelling agent, the quantity of amine functions contributed by the cationic polymer, in the fatty phase, being between 10.8 μmol and 32.4 μmol per gram of fatty phase, and
the aqueous fluid FE comprises at least water and at least one anionic polymer and optionally also at least one hydrophilic texturizing agent.

14. A composition comprising at least one dispersion according to claim 1, optionally in association with at least one physiologically acceptable medium.

15. The composition according to claim 14, said composition being a make-up composition.

16. A non-therapeutic cosmetic treatment method of a keratin material, comprising at least one step to apply to said keratin material at least one dispersion according to claim 1 or at least one composition according to claim 14.

17. The dispersion according to claim 1, wherein the fatty phase also comprises at least one lipophilic gelling agent chosen from among organic or mineral, polymeric or molecular lipophilic gelling agents; fats solid at ambient temperature and pressure; and mixtures thereof.

18. The dispersion according to claim 1, wherein the cationic polymer is a silicone polymer modified by a primary, secondary or tertiary amine function, having the following formula: 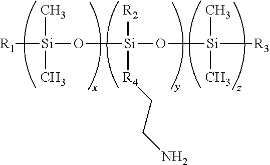
where:
R₁, R₂ and R₅ are each independently OH or CH₃;
R₄ is a group —CH₂— or group —X—NH— where X is a C₃ or C₄ divalent alkylene radical;
x is an integer of between 10 and 5 000;
y is an integer of between 1 and 1 000; and
z is an integer of between 0 and 10.

19. The dispersion according to claim 1, wherein the anionic polymer is chosen from among carbomers or an acrylates/C₁₀₋₃₀ alkyl acrylate Crosspolymer.
